(19) United States
(12) Patent Application Publication
(10) Pub. No.: US 2025/0135205 A1
(43) Pub. Date: May 1, 2025

(54) SYSTEMS AND METHODS FOR EVALUATING SPINAL CORD STIMULATION THERAPY

(71) Applicant: Saluda Medical Pty Ltd, Macquarie Park, New South Wales (AU)

(72) Inventors: Daniel Mark Brounstein, Bloomington, MN (US); Joel Lawrence Barto, Bloomington, MN (US); Quinton John Kocher, Bloomington, MN (US); Dean Michael Karantonis, Macquarie Park (AU); Ian Cameron Gould, Macquarie Park (AU); Joanne Carol Shiels Olecko, Bloomington, MN (US); Daniel John Parker, Macquarie Park (AU); Leah Rebecca Muller, Bloomington, MN (US)

(21) Appl. No.: 18/837,819

(22) PCT Filed: Feb. 14, 2023
(86) PCT No.: PCT/US2023/062604

§ 371 Date: Aug. 12, 2024

Related U.S. Application Data
(60) Provisional application No. 63/267,981, filed on Feb. 14, 2022.

(51) Int. Cl. A61N 1/36; A61N 1/05; G16H 20/40

(52) U.S. Cl. CPC A61N 1/36139; A61N 1/0551; A61N 1/36062; A61N 1/36132; G16H 20/40

(57) ABSTRACT

A method of evaluating spinal cord stimulation (SCS), comprising obtaining neural response data generated by a spinal cord stimulator, where the neural response data describes one or more neural signals evoked in the spinal cord of a patient by applying diagnostic stimuli. One or more evoked compound action potentials (ECAPs) associated with the respective one or more neural signals are detected. The one or more detected ECAPs are processed to generate one or more evaluation metrics, and the one or more evaluation metrics are used to generate a confidence score indicating a degree of effectiveness of SCS on the patient.

The present application claims priority from U.S. Provisional Patent Application No. 63/267,981 filed on 14 Feb. 2022, the contents of which are incorporated herein by reference in their entirety.

### TECHNICAL FIELD

The present invention generally relates to spinal cord stimulation and, more specifically, to rapid evaluation of spinal cord stimulation for individual patients.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation system applies an electrical pulse (stimulus) to neural tissue (fibres, or neurons) in order to generate a therapeutic effect. In general, the electrical stimulus generated by a neuromodulation system evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a system typically comprises an implanted electrical pulse generator, and a power source such as a battery that may be transcutaneously rechargeable by wireless means, such as inductive transfer. An electrode array is connected to the pulse generator and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (towards the head, or rostral) and antidromic (towards the cauda, or caudal) directions. The fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, is therefore necessary to apply stimuli with intensity below a comfort threshold, above which uncomfortable or painful percepts arise due to over-recruitment of Aβ (A-beta) fibres. When recruitment is too large, Aβ fibres produce uncomfortable sensations. Stimulation at high intensity may even recruit Aδ (A-delta) fibres, which are sensory nerve fibres associated with acute pain, cold and pressure sensation. It is therefore desirable to maintain stimulus intensity within a therapeutic range between the recruitment threshold and the comfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Another control problem facing neuromodulation systems of all types is achieving neural recruitment at a sufficient level for therapeutic effect, but at minimal expenditure of energy. The power consumption of the stimulation paradigm has a direct effect on battery requirements which in turn affects the device's physical size and lifetime. For rechargeable systems, increased power consumption results in more frequent charging and, given that batteries only permit a limited number of charging cycles, ultimately this reduces the implanted lifetime of the device.

Attempts have been made to address such problems by way of feedback or closed-loop control, such as using the methods set forth in International Patent Publication No. WO2012155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be generated by a measurement electrode in electrical communication with the recruited neural fibres, and processed to obtain the feedback variable. Based on the response intensity, the intensity of the applied stimulus may be adjusted to maintain the response intensity within a therapeutic range.

It is therefore desirable to accurately detect and record a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO 2012/155183, the content of which is incorporated herein by reference.

However, neural response measurement can be a difficult task as an observed CAP signal component in the measured response will typically have a maximum amplitude in the range of microvolts. In contrast, a stimulus applied to evoke the CAP is typically several volts, and manifests in the measured response as crosstalk of that magnitude. Moreover, stimulus generally results in electrode artefact, which manifests in the measured response as a decaying output of the order of several millivolts after the end of the stimulus. As the CAP signal can be contemporaneous with the stimulus crosstalk and/or the stimulus artefact, CAP measurements present a difficult challenge of measurement amplifier design. For example, to resolve a 10 μV CAP with 1 uV resolution in the presence of stimulus crosstalk of 5 V requires an amplifier with a dynamic range of 134 dB, which is impractical in implantable devices. In practice, many non-ideal aspects of a circuit lead to artefact, and as these aspects mostly result a time-decaying artefact waveform of positive or negative polarity, their identification and elimination can be laborious.

Evoked neural responses are less difficult to detect when they appear later in time than the artefact, or when the signal-to-noise ratio is sufficiently high. The artefact is often restricted to a time of 1-2 ms after the stimulus and so, provided the neural response is detected after this time window, a neural response measurement can be more easily obtained. This is the case in surgical monitoring where there are large distances (e.g. more than 12 cm for nerves conducting at 60 ms⁻¹) between the stimulating and measurement electrodes so that the propagation time from the stimulus site to the measurement electrodes exceeds 2 ms.

However, to characterize the responses from the dorsal column, high stimulation currents are required. Similarly, any implanted neuromodulation device will necessarily be of compact size, so that for such devices to monitor the effect of applied stimuli, the stimulus electrode(s) and measurement electrode(s) will necessarily be in close proximity. In such situations the measurement process must overcome artefact directly.

The difficulty of this problem is further exacerbated when attempting to implement CAP detection in an implanted device. Typical implanted devices have a power budget that permits a limited number, for example in the hundreds or low thousands, of processor instructions per stimulus, in order to maintain a desired battery lifetime. Accordingly, if a CAP detector for an implanted device is to be used regularly (e.g. once a second), then care must be taken that the detector should consume only a small fraction of the power budget.

A functional feedback loop can also produce useful data for live operation and/or post-analysis, such as observed neural response amplitude and applied stimulus intensity. However, device operation at tens of Hz over the course of hours or days quickly produces large volumes of such data which far exceed an implanted device's data storage capacities.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein are systems and methods for the personalized evaluation of spinal cord stimulation as a treatment option. In many embodiments, systems and methods described herein are capable of providing a determination as to whether or not a patient is likely to benefit from spinal cord stimulation and/or to assess the effectiveness of an actual or prescribed spinal cord stimulation therapy program.

According to a first aspect of the present technology, there is provided a method of evaluating spinal cord stimulation (SCS), comprising: obtaining neural response data generated by a spinal cord stimulator, where the neural response data describes one or more neural signals evoked in the spinal cord of a patient by applying diagnostic stimuli: detecting one or more evoked compound action potentials (ECAPs) associated with the one or more neural signals respectively: processing the one or more detected ECAPs to generate one or more evaluation metrics; and using the one or more evaluation metrics to generate a confidence score indicating a degree of effectiveness of SCS on the patient.

In some embodiments, the confidence score indicates a likelihood that the SCS is effective to provide a therapeutic benefit to the patient.

In some embodiments, the first aspect further comprises, in response to the confidence score failing to exceed a predetermined threshold value, providing an indication of: one or more additional activities to further assess whether the SCS is likely to provide a long-term therapeutic effect to the patient: or an instruction or recommendation for explantation of the spinal cord stimulator, and/or one or more leads, from the patient.

In some embodiments, the first aspect further comprises, in response to the confidence score failing to exceed a predetermined threshold value: determining one or more SCS parameters of an SCS program for adjustment; and adjusting the determined one or more SCS parameters by an amount determined, at least in part, by processing the one or more evaluation metrics.

In some embodiments, the one or more evaluation metrics comprises one or more functionality metrics, the one or more functionality metrics including at least one of: a signal-to-noise ratio of a given ECAP:a signal-to-artefact ratio of the given ECAP; and a likelihood metric indicating the confidence with which the given ECAP is detected, wherein the given ECAP is one of the detected ECAPs.

In some embodiments, the one or more evaluation metrics comprises a postural robustness metric.

In some embodiments, the determining the postural robustness metric comprises: (i) providing SCS to the patient via the spinal cord stimulator, wherein the patient is positioned in a candidate posture: (ii) recording, in response to the SCS, a set of neural recruitment magnitudes (NRMs) of the detected ECAPs associated with the neural signals in the spinal cord of the patient in the candidate posture; (iii) determining a first measure of variation to measure variation in the values of the set of NRMs; and (iv) computing a second measure of a plurality of first measures of variation, wherein the plurality of first measures of variation are obtained by iteratively performing steps (i)-(iii) for different candidate postures.

In some embodiments, the one or more evaluation metrics comprises one or more therapy quality metrics, the one or more therapy quality metrics including at least one of: a therapy utilization value: a therapy target level differential value; and a neural activation accuracy score (NAS).

In some embodiments, the determining the NAS comprises: (i) providing SCS to the patient via the spinal cord stimulator, wherein the patient is positioned in a candidate posture: (ii) recording, in response to the SCS, a set of neural recruitment magnitudes (NRMs) of the detected ECAPs associated with the neural signals in the spinal cord of the patient in the candidate posture; and (iii) processing the recorded set of NRMs to compute values of the NAS.

In some embodiments, the steps (i)-(ii) are iteratively repeated for different candidate postures such that step (iii) includes processing respective sets of NRMs recorded for the different candidate postures.

In some embodiments, computing values of the NAS comprises: calculating a representative error value of each recorded set of NRMs relative to corresponding NRMs of a baseline target therapy level; determining a neural activation error (NAE) value by processing one or more of the calculated representative error values; and transforming the NAE value to generate a value of the NAS between zero and a predetermined maximum value.

In some embodiments, the representative error value of the set of NRMs is a root mean square error (RMSE) value.

In some embodiments, the NAE value is normalized or scaled relative to a predetermined feedback variable.

In some embodiments, the predetermined maximum value is 100.

In some embodiments, a confidence sub-score is generated for each of the one or more therapy quality metrics of the evaluation metrics.

In some embodiments, generating the confidence score comprises calculating a weighted sum of confidence sub-scores.

In some embodiments, generating the confidence score comprises: comparing one or more therapy quality metric values to one or more corresponding predetermined metric thresholds; classifying each of the one or more therapy quality metric values as one of a plurality of predetermined efficacy categories based on the comparing; and determining the confidence score based on the classifications of the one or more therapy quality metric values.

In some embodiments, the confidence score is determined as one of a set of predetermined confidence score values based on a number of classifications of the one or more therapy quality metric values in each efficacy category.

In some embodiments, generating the confidence score comprises: performing one or more evaluation tests against the one or more therapy quality metric values, each evaluation test involving performing a series of one or more comparisons between a therapy quality metric value and one or more corresponding threshold values; and setting or adjusting the confidence score in response to the outcome of the one or more comparisons of each evaluation test.

In some embodiments, the steps of performing evaluation tests and setting or adjusting the confidence score are organized according to a decision tree to prioritize a relative degree of importance of the therapy quality metrics.

In some embodiments, generating the confidence score further comprises, in response to a therapy quality metric value failing to exceed the corresponding threshold value, setting the confidence score to a minimum value.

In some embodiments, the confidence score is generated by further using one or more additional metrics selected from the group consisting of: a dermatome activation metric, a patient feedback metric, a gait metric, heart rate variability, and pupil dilation.

In some embodiments, the confidence score is generated using a machine learning mode.

In some embodiments, the confidence score is generated by calculating a sum of the values of the one or more evaluation metrics.

According to a second aspect of the present technology, there is provided a spinal cord stimulation evaluator, comprising: a processor: an input/output (I/O) interface configured to at least receive neural response data; and a memory, the memory containing a spinal cord stimulation evaluation application configured to direct the processor to perform the method of evaluating spinal cord stimulation of the first aspect of the present technology.

According to a third aspect of the present technology, there is provided a system for evaluating spinal cord stimulation (SCS), comprising: a stimulator device comprising an electrode array and a pulse generator, the stimulator device configured to: apply, via the pulse generator, diagnostic stimuli to the spinal cord of a patient via one or more stimulus electrodes of the electrode array; and measure, via one or more measurement electrodes of the electrode array, values of one or more neural response signals evoked by the diagnostic stimuli; and at least one processor configured to: receive neural response data indicating values of the one or more neural response signals: detect one or more evoked compound action potentials (ECAPs) associated with the one or more neural signals respectively: process the one or more detected ECAPs to generate one or more evaluation metrics; and use the one or more evaluation metrics to generate a confidence score indicating a degree of effectiveness of SCS on the patient.

In some embodiments, the at least one processor is further configured to, in response to the confidence score failing to exceed a predetermined threshold value, provide an indication of: one or more additional activities to further assess whether the SCS is likely to provide a long-term therapeutic effect to the patient: or an instruction or recommendation for explantation of the spinal cord stimulator, and/or one or more leads, from the patient.

In some embodiments, the at least one processor is further configured to, in response to the confidence score failing to exceed a predetermined threshold value: determine one or more SCS parameters of an SCS program for adjustment; and adjust the determined one or more SCS parameters by an amount determined, at least in part, by processing the one or more evaluation metrics.

In some embodiments, determining the postural robustness metric comprises: (i) providing, by the stimulator device, SCS to the patient, wherein the patient is positioned in a candidate posture: (ii) recording, by the at least one processor and in response to the SCS, a set of neural recruitment magnitudes (NRMs) of the detected ECAPs associated with the neural signals in the spinal cord of the patient in the candidate posture: (iii) determining, by the at least one processor, a first measure of variation to measure variation in the values of the set of NRMs; and (iv) computing, by the at least one processor, a second measure of variation of a plurality of first measures of variation, wherein the plurality of first measures of variation are obtained by iteratively performing steps (i)-(iii) for different candidate postures.

In some embodiments, determining the NAS comprises: (i) providing, by the stimulator device, SCS to the patient, wherein the patient is positioned in a candidate posture: (ii) recording, by the at least one processor and in response to the SCS, a set of neural recruitment magnitudes (NRMs) of the detected ECAPs associated with the neural signals in the spinal cord of the patient in the candidate posture; and (iii) processing, by the at least one processor, the recorded set of NRMs to compute values of the NAS.

In some embodiments, computing values of the NAS comprises: calculating, by the at least one processor, a representative error value of each recorded set of NRMs relative to corresponding NRMs of a baseline target therapy level: determining, by the at least one processor, a neural activation error (NAE) value by processing one or more of the calculated representative error values; and transforming, by the at least one processor, the NAE value to generate a value of the NAS between zero and a predetermined maximum value.

In some embodiments, generating the confidence score comprises: comparing, by the at least one processor, one or more therapy quality metric values to one or more corresponding predetermined metric thresholds: classifying, by the at least one processor, each of the one or more therapy quality metric values as one of a plurality of predetermined efficacy categories based on the comparing; and determining, by the at least one processor, the confidence score based on the classifications of the one or more therapy quality metric values.

In some embodiments, generating the confidence score comprises: performing, by the at least one processor, one or more evaluation tests against the one or more therapy quality metric values, each evaluation test involving performing a series of one or more comparisons between a therapy quality metric value and one or more corresponding threshold values; and setting or adjusting, by the at least one processor, the confidence score in response to the outcome of the one or more comparisons of each evaluation test.

In some embodiments, generating the confidence score further comprises, in response to a therapy quality metric value failing to exceed the corresponding threshold value, setting, by the at least one processor, the confidence score to a minimum value.

In some embodiments, the confidence score is generated by calculating, by the at least one processor, a sum of the values of the one or more evaluation metrics.

In some embodiments, the system for evaluating spinal cord stimulation of the third aspect of the present technology is configured to perform the method of evaluating spinal cord stimulation of the first aspect of the present technology.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 schematically illustrates an implanted spinal cord stimulator, according to one implementation of the present technology;

FIG. 2 is a block diagram of the stimulator of FIG. 1:

FIG. 3 is a schematic illustrating interaction of the implanted stimulator of FIG. 1 with a nerve:

FIG. 4a illustrates an idealised activation plot for one posture of a patient undergoing neurostimulation:

FIG. 4b illustrates the variation in the activation plots with changing posture of the patient:

FIG. 5 is a schematic illustrating elements and inputs of a closed-loop neurostimulation system, according to one implementation of the present technology:

FIG. 6 illustrates the typical form of an electrically evoked compound action potential (ECAP) of a healthy subject:

FIG. 7 is a block diagram of a neuromodulation therapy system including the implanted stimulator of FIG. 1 according to one implementation of the present technology:

FIG. 8 is a block diagram illustrating the data flow of a neuromodulation therapy system such as the system of FIG. 7;

FIG. 9 schematically illustrates a spinal cord stimulation evaluation system in accordance with an embodiment of the invention.

FIG. 10 is a block diagram of a spinal cord stimulation evaluator in accordance with an embodiment of the invention.

FIG. 11A is a flow chart for a first spinal cord stimulation evaluation process in accordance with an embodiment of the invention.

FIG. 11B is a flow chart for a second spinal cord stimulation evaluation process in accordance with an embodiment of the invention.

FIG. 12 is a graphical illustration of a spinal cord stimulation evaluation process in accordance with an embodiment of the invention.

FIGS. 13A, B, and C, are charts illustrating a lack of an ECAP, presence of an ECAP with artefact, and presence of an ECAP with little artefact in a neural signal, respectively, in accordance with an embodiment of the invention.

FIG. 14 is a flow chart for a process for calculating ECAP-derived patient functionality metrics in accordance with an embodiment of the invention.

FIG. 15 is a flow chart for a process for calculating an ECAP likelihood in accordance with an embodiment of the invention.

FIG. 16 is a flow chart for a process for computing activation plot quality metrics in accordance with an embodiment of the invention.

FIG. 17 is a chart illustrating example changes in feedback variable based on patient posture in accordance with an embodiment of the invention.

FIG. 18 is a flow chart for a process for evaluating postural robustness of spinal cord stimulation in accordance with an embodiment of the invention.

FIG. 19A is a flow chart for a process for computing therapy quality metrics in accordance with an embodiment of the invention.

FIG. 19B is a graph illustrating a measure of neural activation accuracy in accordance with an embodiment of the invention.

FIG. 19C is a flow chart for a process for determining values of a therapy quality metric in accordance with an embodiment of the invention.

FIG. 20A is a chart illustrating example therapy quality metrics in accordance with an embodiment of the invention.

FIG. 20B is a graph illustrating activation level deviation for an exemplary metric calculation in accordance with an embodiment of the invention.

FIG. 20C is a graph illustrating a sigmoid transformation function used to generate neural activation accuracy scores from the deviations of FIG. 20B.

FIG. 21 is a graphical example score calculation process in accordance with an embodiment of the invention.

FIG. 22A is a flow chart for a process for determining a confidence score from values of one or more therapy quality metrics in accordance with an embodiment of the invention.

FIG. 22B is illustrates an exemplary table of classifications of therapy quality metric values in accordance with the process of FIG. 22A.

FIG. 22C is a flow chart for an example decision tree process for determining a confidence score from values of therapy quality metrics in accordance with an embodiment of the invention.

### DETAILED DESCRIPTION OF THE PRESENT TECHNOLOGY

Spinal cord stimulation (SCS) is a procedure which can reduce or eliminate chronic pain. However, SCS does not provide the same degree of therapeutic benefit for every patient. Furthermore, it is rarely immediately apparent whether a patient will receive a sufficiently beneficial therapeutic effect, or any therapeutic effect at all, from the application of SCS.

The uncertainty associated with the potential therapeutic outcome of SCS is problematic because treatment via SCS requires a surgically invasive procedure to be performed on the patient. Currently, SCS patients undergo lengthy trial periods which are both cumbersome and have significant risks. For a period of one to two weeks, electrodes are surgically implanted into the patient's spine, but the pulse generator (stimulator) remains external to the body. Therefore, during this trial period, electrode leads are exiting the body via an open tract. Binders and other bandages can be used to keep the pulse generator and leads from moving. In the event that the leads are tugged, the electrode placement can be disrupted, or in the most catastrophic scenarios, tissue can be torn or damaged. Further, during the trial many patients are required to restrict their movement as to reduce these risks.

More generally, there exists a need to assess or evaluate the efficacy of SCS for a patient, including in scenarios where SCS is presently being performed, or is planned to be performed, on the patient in accordance with a SCS program (i.e., post completion of the stimulator implantation). Conventional approaches to assessing SCS are largely qualitative resulting in difficulties with performing accurate adjustments to the SCS treatment program. Furthermore, there are difficulties in determining or quantifying unexpected or otherwise adverse outcomes of an SCS program (e.g., where a patient is experiencing increased pain and/or decreased function in response to the SCS). It is desirable, at least in the aforementioned contexts, to determine the likely or actual effectiveness of SCS therapy for a patient, and to thereby address one or more drawbacks of the prior art, or other drawbacks, or to at least provide a useful alternative.

Systems and methods described herein enable the evaluation of the therapeutic effectiveness of SCS for a patient, by: (i) recording neural response data indicating a neural response of a patient to diagnostic SCS: (ii) calculating one or more evaluation metrics derived from a signal window of the neural response data; and (iii) using the one or more evaluation metrics to define a confidence score indicating a degree of effectiveness of SCS on the patient. The evaluation may be in the form of a prediction indicating whether SCS is likely to provide a long-term therapeutic effect to the patient (e.g., prior to completion of the surgical implantation procedure). In such scenarios, the described systems and methods attempt to eliminate the lengthy trial periods, and instead provide an evaluation period that is substantially shorter than existing periods (e.g., less than 24 hours).

In other embodiments, the SCS evaluation may be performed in association with a SCS treatment program currently prescribed for the patient or intended for prescription to the patient (i.e., following completion of the implantation). In such scenarios, the described systems and methods attempt to determine whether the SCS treatment program is providing, or is likely to provide, an effective therapeutic benefit to the patient, or whether the program needs adjustment.

In many embodiments, systems and methods described herein obtain and utilize a number of different signals and patient responses which are used to calculate one or more confidence scores. The response of the patient to stimulus signals, as applied to the spinal cord by a stimulator, is a set of neural response signals which are described by corresponding neural response data. Further signals and/or parameters may be detected in association with the neural response signals, including one or more evoked compound action potentials (ECAPs). As discussed above, an ECAP is a measure of electrical response from tissue to stimulation. In particular, an ECAP is the sum of the contributions from all nerve fibres that respond to an electrical stimulus.

In numerous embodiments, the evaluation metrics are generated from the waveform of ECAPs recorded from the patient during diagnostic stimulation applied by the stimulator. ECAP-derived functionality metrics include: the signal to noise ratio (SNR), the signal to artefact ratio (SAR), a detection confidence of the ECAP (ECAP likelihood), one or more morphological stability metrics, the SAR coefficient of variance (SAR CoV), a signal quality indicator, and activation plot quality metrics.

In various embodiments, the evaluation metrics include additional metrics determined from the patient independently of the detection of any ECAP associated with the one or more neural signals. For example, the additional metrics may include one or more of dermatome activation, patient feedback, patient gait, heart rate variability, and pupil dilation.

In numerous embodiments, ECAP morphology is used in calculating the confidence score. In a variety of embodiments, ECAP morphology includes axonal properties derived from the ECAP such as (but not limited to) conduction velocity and chronaxie.

Values of the one or more evaluation metrics may be generated from a diagnostic evaluation session, in which the spinal cord stimulator is configured to apply diagnostic stimulation to the patient. The values of the evaluation metrics are used to generate at least one confidence score indicating a degree of the effectiveness of the SCS on the patient.

The type and delivery of the diagnostic SCS may vary based on a number of factors including the evaluation scenario and the characteristics of the patient. For example, the diagnostic stimulation may include sub-threshold, supra-threshold, variable field stimulation (involving variations to pulse width, amplitude, and/or frequency of the stimulus signal), posture-based stimulation and any other stimulation modality that is available to the clinician or other user of the system.

In one evaluation scenario, the diagnostic SCS is delivered with the goal of determining whether SCS is likely to provide a long-term therapeutic effect to the patient (e.g., prior to completing a full implantation of a stimulator device and associated leads). For example, in response to the confidence score exceeding a predetermined threshold, the clinician may choose to complete the implantation process thereby eliminating the need for the patient to undergo a long trial period.

In response to the confidence score failing to exceed the threshold, the systems and methods may provide an indication of additional activities to further assess whether the SCS is likely to provide a long-term therapeutic effect to the patient. For example, the additional activities may include performing further trials or diagnostic procedures on the patient to further evaluate the potential therapeutic benefit of the SCS. Alternatively, the systems and methods may provide an instruction or recommendation for a clinician to explant the spinal cord stimulator from the patient (i.e., in the case that the SCS is deemed unlikely to have a significant therapeutic effect for the patient). A clinician may therefore utilize the output of the systems to improve treatment outcomes for the patient.

In another evaluation scenario, the diagnostic SCS is associated with a particular SCS therapy that is presently being performed on, or intended to be performed on, the patient as part of an SCS program, post completion of stimulator implantation. The diagnostic SCS may involve stimulation using one or more of the SCS program parameters such as to replicate the actual or intended effect of the SCS program. Alternatively, the diagnostic SCS may correspond to a portion of SCS performed on the patient by execution of the SCS program.

In some embodiments, the evaluation metrics are processed to provide an indication of the viability for the patient to have a stable therapeutic effect using open and/or closed loop stimulation. Open loop stimulation in the present specification refers to operation of a SCS device to apply SCS with a fixed stimulus intensity, where the intensity is often predetermined prior to the commencement of the SCS therapy. Closed loop stimulation refers to SCS devices which automatically respond to sensed changes in neural activation in order to maintain therapeutic effect. The operation of the SCS device as either open—or closed-loop is determined by a SCS program mode parameter of a corresponding SCS program. In some embodiments, the SCS program mode parameter is adjusted by processing one or more evaluation metrics to determine a predicted or actual improvement in the efficacy of the SCS in the open or closed loop modes. For example, a postural robustness metric may be calculated by measuring variation in a set of neural recruitment magnitudes across a number of tests in which the patient is positioned in different postures.

In numerous embodiments, the evaluation metrics include one or more therapy quality metrics. Therapy quality metrics provide an objective indication of the efficacy of SCS therapy on a patient by comparing parameters associated with a proposed or actual prescribed SCS therapy to predetermined values that represent a baseline or expected level. Some therapy quality metrics are derived from one or more parameters of the detected ECAPs, such as for example a therapy target level value and a neural activation accuracy score (NAS). For example, the one or more parameters of the detected ECAPs may include a set of neural recruitment magnitudes (NRMs) from ECAPs detected in response to SCS applied to the patient placed in a candidate posture. Postural effects are accounted for by processing respective sets of NRMs recorded for different candidate postures to determine the therapy quality metric value(s). Other therapy quality metrics are derived independently of the one or more neural signals described by the neural response data, such as for example a therapy utilization value.

In the described embodiments, the confidence score is generated from the values of the one or more evaluation metrics. The one or more evaluation metrics may include ECAP-derived functionality metrics, postural robustness metrics or scores, and/or therapy quality metrics. For example, the confidence score may be calculated as a weighted sum of one or more functionality metrics and one or more therapy quality metrics derived from the detected ECAPs. In some embodiments, a confidence score is generated from one or more sub-scores produced for each evaluation metric. Corresponding threshold values are defined for each generated score to enable a patient-specific assessment of the effectiveness of SCS in accordance with the techniques described herein.

In some embodiments, the one or more evaluation metrics used to generate the confidence score are selected dynamically depending on the purpose of the evaluation. For example, in scenarios where the evaluation is conducted for the purpose of predicting a likelihood of long-term therapeutic benefit to decide whether to complete implantation, the evaluation metrics may comprise primarily ECAP-derived functionality metrics (i.e., in order to facilitate conducting diagnostic trials and achieving a decision outcome within a short period of time). In other scenarios where the evaluation is performed to assess the efficacy of a prescribed SCS program (i.e., post-implantation of the stimulator) the evaluation metrics may include therapy quality metrics. However, it will be appreciated that any of the therapy quality metrics may also be used in the former scenario at the discretion of the clinician.

The generation of a confidence score from one or more evaluation metrics provides a means of assessing SCS efficacy that is advantageous compared to other approaches that utilize the values of the evaluation metrics (or their statistical profile) directly. For example, the confidence scores generated according to the described embodiments are applicable to indicate effectiveness of the SCS relative to separate success and failure thresholds, which enables more than a binary decision outcome in relation to SCS efficacy. Using sub-scores for individual therapy quality metric values enables the confidence score to be calculated efficiently based on a degree of relative importance of each evaluation metric to the assessment (e.g., using categorization tables and/or decision trees).

The use of therapy quality metrics is advantageous in providing an objective measure of the efficacy of a SCS therapy program that is based on the consistency and level of the neural activation achieved by the patient undergoing the SCS therapy. This enables a prediction or assessment of SCS viability that is based on the patient's inherent potential for SCS and their actual therapeutic use case. As a result of the confidence score, and optionally individual sub-scores for the metric values, a SCS program prescribed to, or developed for, the patient may be adjusted leading to an improved SCS therapeutic outcome.

Spinal cord stimulation systems as well as systems and methods for their evaluation are discussed herein.

### Spinal Cord Stimulators

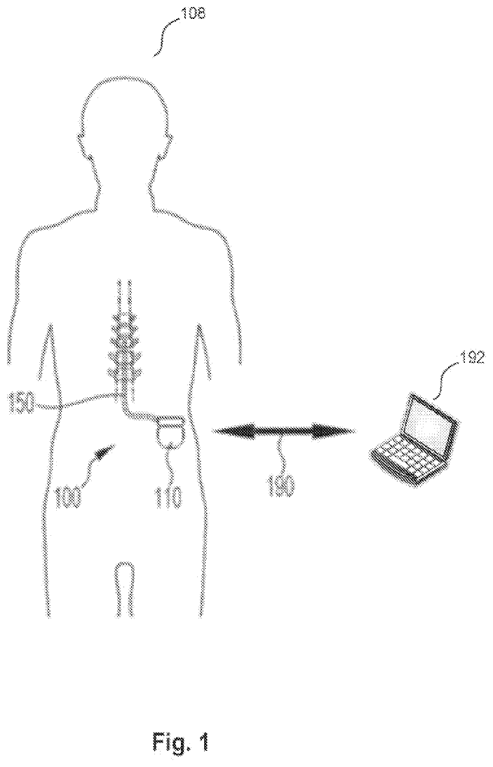

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as a flank or sub-clavicular. Stimulator 100 further comprises an electrode array 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

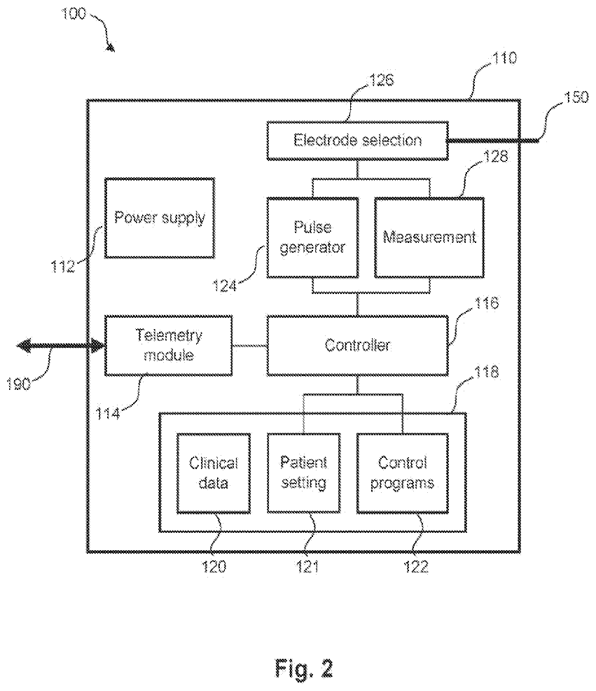

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive and inductive transfer, may be used by telemetry module 114 to transfer power and/or data to and from the electronics module 110 via communications channel 190. Module controller 116 has an associated memory 118 storing one or more of clinical data 120, patient settings 121, control programs 122, and the like. Controller 116 controls a pulse generator 124 to generate stimuli, such as in the form of pulses, in accordance with the patient settings 121 and control programs 122. Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

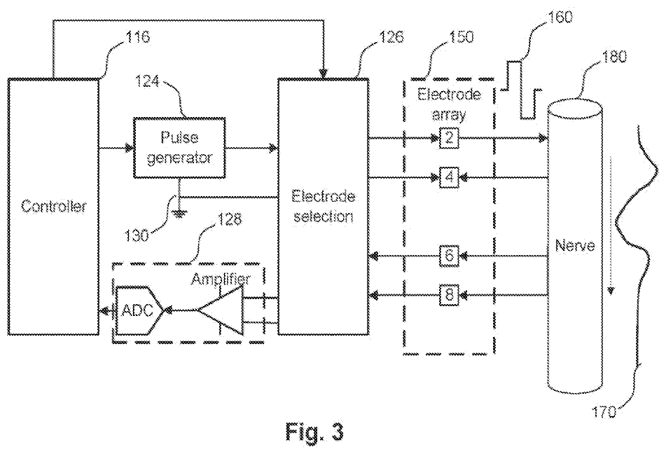

FIG. 3 is a schematic illustrating interaction of the implanted stimulator 100 with a nerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulus electrode 2 of electrode array 150 through which to deliver a pulse from the pulse generator 124 to surrounding tissue including nerve 180. A pulse may comprise one or more phases, e.g. a biphasic stimulus pulse 160 comprises two phases. Electrode selection module 126 also selects a return electrode 4 of the electrode array 150 for stimulus charge recovery in each phase, to maintain a zero net charge transfer. Because a given electrode may act as both a stimulus and a return electrode over a complete multiphasic stimulus pulse, both electrodes are generally referred to as stimulus electrodes. The use of two electrodes in this manner for delivering and recovering current in each stimulus phase is referred to as bipolar stimulation. Alternative embodiments may apply other forms of bipolar stimulation, or may use a greater number of stimulus electrodes. Electrode selection module 126 is illustrated as connecting to a ground 130 of the pulse generator 124 to enable stimulus charge recovery via the return electrode 4. However, other connections for charge recovery may be used in other implementations.

Delivery of an appropriate stimulus from stimulus electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the stimulus electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus configuration is found which evokes paraesthesia in a location and of a size which is congruent with the area of the patient's body affected by pain, the clinician nominates that configuration for ongoing use.

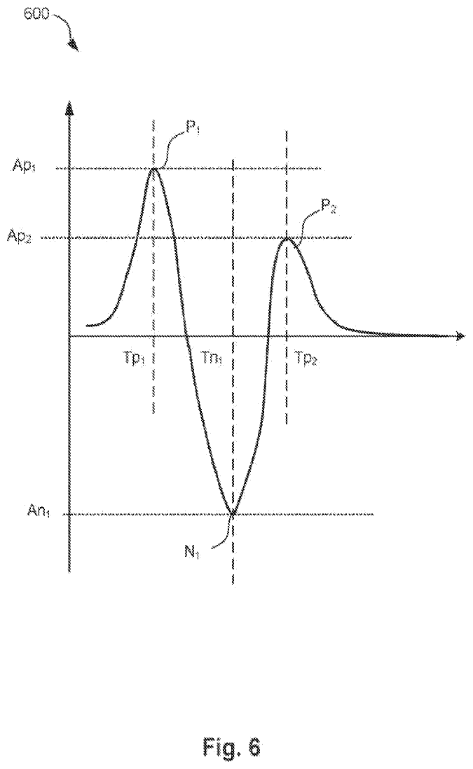

FIG. 6 illustrates the typical form of an ECAP 600 of a healthy subject, as recorded at a single measurement electrode referenced to the system ground 130. The shape and duration of the ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be parametrised by any suitable parameter(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to detect the existence and measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as measurement electrode 6 and measurement reference electrode 8, whereby the electrode selection module 126 selectively connects the chosen electrodes to the inputs of the measurement circuitry 128. Thus, signals sensed by the measurement electrodes 6 and 8 are passed to the measurement circuitry 128, which may comprise a differential amplifier and an analog-to-digital converter (ADC), as illustrated in FIG. 3. The measurement circuitry 128 for example may operate in accordance with the teachings of International Patent Application Publication No. WO2012155183 by the present applicant, the content of which is incorporated herein by reference.

Signals sensed by the measurement electrodes 6, 8 and processed by measurement circuitry 128 are further processed by an ECAP detector implemented within controller 116 to obtain information regarding the effect of the applied stimulus upon the nerve 180. In some implementations, the sensed signals are processed by the ECAP detector in a manner which extracts and stores one or more parameters from each evoked neural response or group of evoked neural responses contained in the sensed signal. In one such implementation, the parameter comprises a peak-to-peak ECAP amplitude in microvolts (μV). For example, the neural responses may be processed by the ECAP detector to determine the peak-to-peak ECAP amplitude in accordance with the teachings of International Patent Publication No. WO 2015/074121, the contents of which are incorporated herein by reference. Alternative implementations of the ECAP detector may extract and store an alternative parameter from the neural response, or may extract and store two or more parameters from the neural response.

Stimulator 100 applies stimuli over a potentially long period such as days, weeks, or months and during this time may store parameters of neural responses, stimulation settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more parameters such as a measure of the amplitude of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

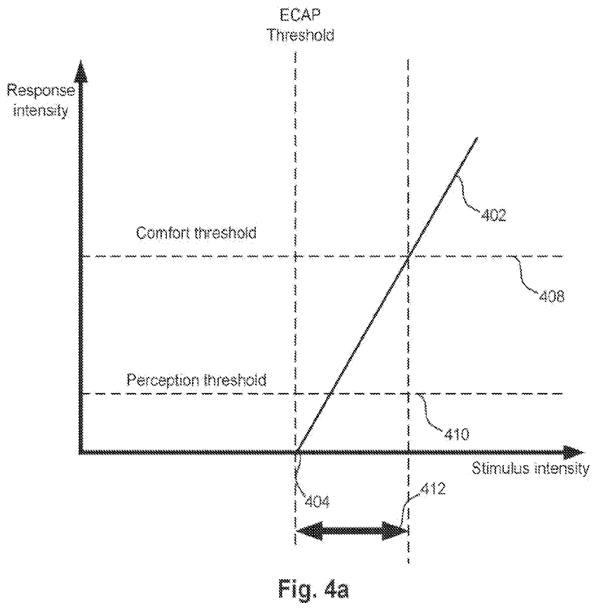

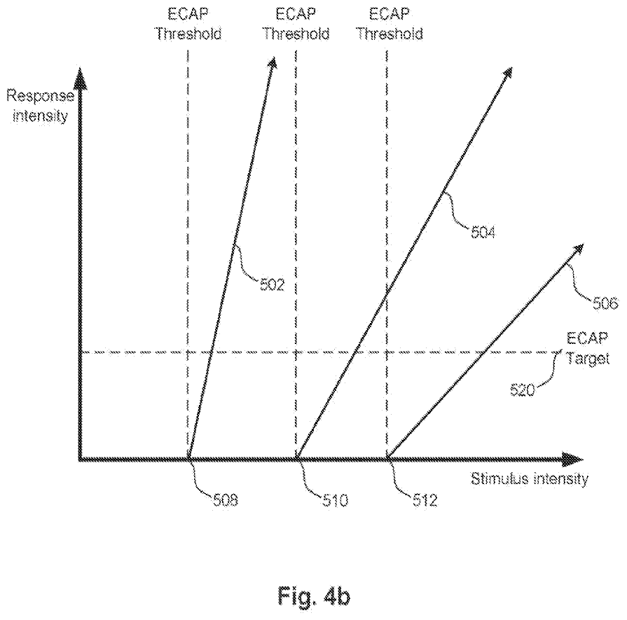

An activation plot, or growth curve, is an approximation to the relationship between stimulus intensity (e.g. an amplitude of the current pulse 160) and intensity of neural response 170 resulting from the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment: if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled as:

$$y = \{ \begin{matrix}S ( s − T ) , & s \geq T \\ 0 , & s < T\end{matrix} \qquad ( 1 )$$

where s is the stimulus intensity, y is the ECAP amplitude, Tis the ECAP threshold and S is the slope of the activation plot (referred to herein as the patient sensitivity). The slope S and the ECAP threshold T are the key parameters of the activation plot 402.

FIG. 4a also illustrates a comfort threshold 408, which is an ECAP amplitude above which the patient 108 experiences uncomfortable or painful stimulation. FIG. 4 also illustrates a perception threshold 410. The perception threshold 410 corresponds to an ECAP amplitude that is perceivable by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus intensity within a therapeutic range. A stimulus intensity within a therapeutic range is above the ECAP threshold 404 and evokes an ECAP amplitude that is below the comfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the neurons. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the slope of the activation plot also changes, as indicated by the varying slopes of activation plots 502, 504, and 506. In general, as the distance between the stimulus electrodes and the spinal cord increases, the ECAP threshold increases and the slope of the activation plot decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more extracted ECAP parameters. In one implementation, the device may adjust the stimulus intensity to maintain the extracted ECAP amplitude at a target response intensity. For example, the device may calculate an error between a target ECAP value and a measured ECAP amplitude, and adjust the applied stimulus intensity to reduce the error as much as possible, such as by adding the scaled error to the current stimulus intensity. A neuromodulation device that operates by adjusting the applied stimulus intensity based on an extracted ECAP parameter is said to be operating in closed-loop mode and will also be referred to as a closed-loop neural stimulus (CLNS) device. By adjusting the applied stimulus intensity to maintain the extracted ECAP amplitude at an appropriate target response intensity, such as an ECAP target 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is characterised by multiple parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The evoked neural response (e.g. an ECAP) is detected, and its amplitude measured by the CLNS device and compared to the target response intensity.

The measured neural response amplitude, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

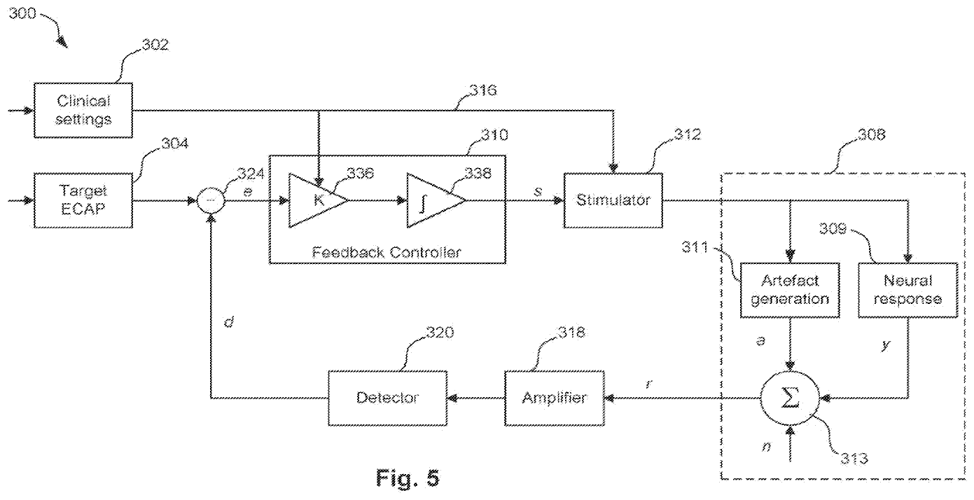

FIG. 5 is a schematic illustrating elements and inputs of a closed-loop neurostimulation system (CLNS) 300, according to one implementation of the present technology. The system 300 comprises a stimulator 312 which converts a stimulus intensity parameter (for example a stimulus current amplitude) s, in accordance with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrode. Various sources of noise n, as well as the artefact a, may add to the evoked response y at the summing element 313 to form the sensed signal r, including electrical noise from external sources such as 50 Hz mains power: electrical disturbances produced by the body such as neural responses evoked not by the device but by other causes such as peripheral sensory input, EEG, EMG, and electrical noise from amplifier 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

The sensed signal r (including evoked neural response, artefact, and noise) is amplified by the signal amplifier 318 and then processed by the ECAP detector 320. The ECAP detector 320 outputs a measured neural response intensity d. In one implementation, the neural response intensity comprises an ECAP value. The comparator 324 compares the measured response intensity d to the target ECAP value as set by the target ECAP controller 304 and provides an indication of the difference between the measured response intensity d and the target ECAP value. This difference is the error value, e. The error value e is input into the feedback controller 310.

The feedback controller 310 calculates an adjusted stimulus intensity parameter, s, with the aim of maintaining a measured response intensity d equal to the target ECAP value. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, an adjustment 8s to the current stimulus intensity parameter s may be computed by the feedback controller 310 as

$$s = f Kedt \qquad ( 2 )$$

A target ECAP value is input to the comparator 324 via the target ECAP controller 304. In one embodiment, the target ECAP controller 304 provides an indication of a specific target ECAP value. In another embodiment, the target ECAP controller 304 provides an indication to increase or to decrease the present target ECAP value. The target ECAP controller 304 may comprise an input into the neural stimulus device, via which the patient or clinician can input a target ECAP value, or indication thereof. The target ECAP controller 304 may comprise memory in which the target ECAP value is stored, and provided to the comparator 324.

A clinical settings controller 302 provides clinical parameters to the system, including the gain K for the gain element 336 and the stimulation parameters for the stimulator 312. The clinical settings controller 302 may be configured to adjust the gain K of the gain element 336 to adapt the feedback loop to patient sensitivity. The clinical settings controller 302 may comprise an input into the neural stimulus device, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 10 kHz). As the ECAP detector 320 is linear, only the stimulus clock affects the dynamics of the CLNS 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

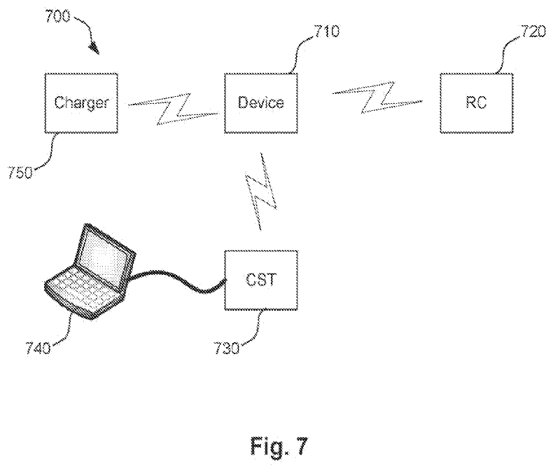

FIG. 7 is a block diagram of a neuromodulation system 700. The neuromodulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation: adjustment of stimulus intensity or target neural response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

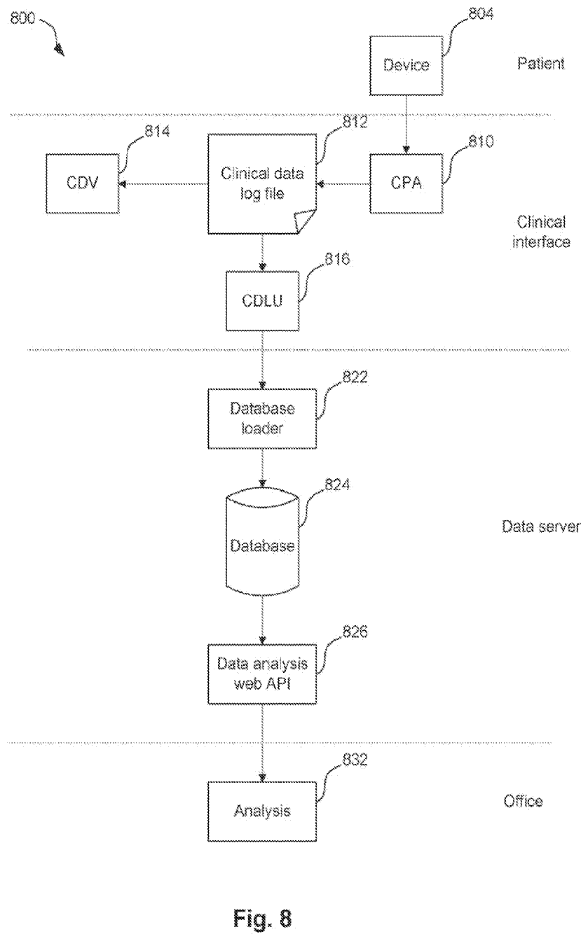

FIG. 8 is a block diagram illustrating the data flow 800 of a neuromodulation therapy system such as the system 700 of FIG. 7 according to one implementation of the present technology. Neuromodulation device 804, once implanted within a patient, applies stimuli over a potentially long period such as weeks or months and records neural responses, stimulation settings, paraesthesia target level, and other operational parameters, discussed further below. Neuromodulation device 804 may comprise a Closed-Loop Stimulator (CLS), in that the recorded neural responses are used in a feedback arrangement to control stimulation settings on a continuous or ongoing basis. To effect suitable SCS therapy, neuromodulation device 804 may deliver tens, hundreds or even thousands of stimuli per second, for many hours each day. The feedback loop may operate for most or all of this time, by obtaining neural response recordings following every stimulus, or at least obtaining such recordings regularly. Each recording generates a feedback variable such as a measure of the amplitude of the evoked neural response, which in turn results in the feedback loop changing the stimulation parameters for a following stimulus. Neuromodulation device 804 thus produces such data at a rate of tens or hundreds of Hz, or even kHz, and over the course of hours or days this process results in large amounts of clinical data. This is unlike past neuromodulation devices such as open-loop SCS devices which lack any ability to record any neural response.

When brought in range with a receiver, neuromodulation device 804 transmits data, e.g. via telemetry module 114, to a clinical programming application (CPA) 810 installed on a clinical interface. In one implementation, the clinical interface is the CI 740 of FIG. 7. The data can be grouped into two main sources: (1) Data collected in real-time during a programming session: (2) Data downloaded from a stimulator after a period of non-clinical use by a patient. CPA 810 collects and compiles the data into a clinical data log file 812.

All clinical data transmitted by the neuromodulation device 804 may be compressed by use of a suitable data compression technique before transmission by telemetry module 114 and/or before storage into the memory 118 to enable storage by neuromodulation device 804 of higher resolution data. This higher resolution allows neuromodulation device 804 to provide more data for post-analysis and more detailed data mining for events during use. Alternatively, compression enables faster transmission of standard-resolution clinical data.

The clinical data log file 812 is manipulated, analysed, and efficiently presented by a clinical data viewer (CDV) 814 for field diagnosis by a clinician, field clinical engineer (FCE) or the like. CDV 814 is a software application installed on the Clinical Interface (CI). In one implementation, CDV 814 opens one Clinical Data Log file 812 at a time. CDV 814 is intended to be used in the field to diagnose patient issues and optimise therapy for the patient. CDV 814 may be configured to provide the user or clinician with a summary of neuromodulation device usage, therapy output, and errors, in a simple single-view page immediately after log files are compiled upon device connection.

Clinical Data Uploader 816 is an application that runs in the background on the CI, that uploads files generated by the CPA 810, such as the clinical data log file 812, to a data server. Database Loader 822 is a service which runs on the data server and monitors the patient data folder for new files. When Clinical Data Log files are uploaded by Clinical Data Uploader 816, database loader 822 extracts the data from the file and loads the extracted data to Database 824.

The data server further contains a data analysis web API 826 which provides data for third-party analysis such as by the analysis module 832, located remotely from the data server. The ability to obtain, store, download and analyse large amounts of neuromodulation data means that the present technology can: improve patient outcomes in difficult conditions: enable faster, more cost effective and more accurate troubleshooting and patient status; and enable the gathering of statistics across patient populations for later analysis, with a view to diagnosing aetiologies and predicting patient outcomes.

### SCS Evaluation

In many embodiments, spinal cord stimulation systems as discussed herein can determine or evaluate the effectiveness of SCS on a patient. The degree of effectiveness may be an indication of: (1) a likelihood of a long-term positive therapeutic benefit from a proposed or potential SCS treatment (i.e., in a “SCS trial” scenario); or (2) a degree of effectiveness of a SCS program for the patient (i.e., in a “SCS program assessment” scenario). In the SCS trial scenario, the proposed systems and methods rapidly determine whether or not a patient will receive sufficient therapeutic benefit of SCS therapy to justify complete implantation. In many embodiments, the determination can be made within 24 hours, and in various embodiments, within 3 hours. Rapid determination provides significant benefits to both the patient and medical staff, as the patient does not need a long and uncomfortable trial period, and fewer hospital resources need to be allocated than would conventionally be used to monitor such a trial.

In the SCS program assessment scenario, the proposed systems and methods may be used, for example, to decide whether to approve or continue a SCS program, or to adjust one or more parameters of the program to improve the actual, or expected, therapeutic result for the patient. SCS systems which can provide the above advantageous effects are discussed herein.

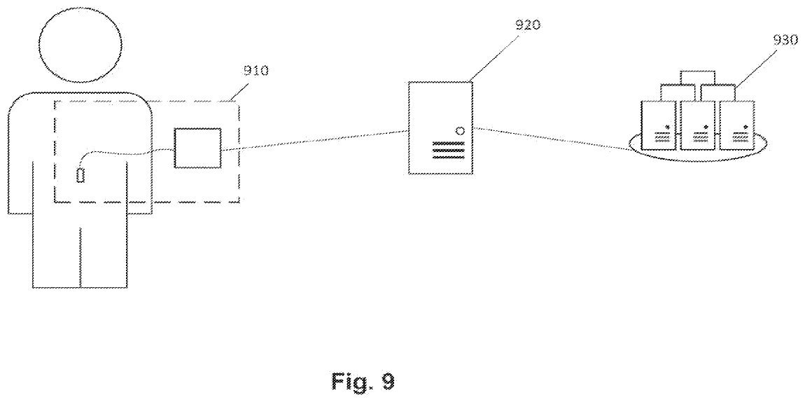

FIG. 9 illustrates a SCS evaluation system 900 in accordance with an embodiment of the invention. SCS evaluation system 900 includes an SCS device 910 which is communicatively coupled to an SCS evaluator 920. In numerous embodiments, the electronics component of the SCS device 910 (the stimulator) is not implanted into the patient and remains external during evaluation. In other embodiments, the system 900 is configured to evaluate a program of SCS therapy for which the SCS device 910 is presently configured to deliver to the patient via the stimulator (i.e., post implantation of the stimulator into the patient).

In many embodiments, transmission of data between the SCS device 910 and the SCS evaluator 920 occurs wirelessly, and may involve intermediate communication devices such as (but not limited to) transceivers, cell phones, wearable computing devices, and/or any other device capable of receiving information from the stimulator of device 910 and transferring it to the SCS evaluator 920 as appropriate to the requirements of specific applications of embodiments of the invention. Alternatively, or in addition, transmission may occur via a wired connection. Further, in numerous embodiments, the SCS device 910 and SCS evaluator 920 can be implemented on the same platform (i.e. on a device integrating the SCS device 910 and SCS evaluator 920).

System 900 also includes a data server system 930 configured to receive data from the SCS evaluator 920. In numerous embodiments, data server system 930 performs at least part of the spinal cord stimulator evaluation processes described herein in lieu or in conjunction with the SCS evaluator 920. In various embodiments, data server system 930 provides updates to the SCS evaluator 920. For example, data may be transmitted between data server systems, SCS evaluators, pulse generators, and/or communication devices by any type of network, including (but not limited to) wireless networks, wired networks, and/or any combination of networks as appropriate to the requirements of specific applications of embodiments of the invention. While a particular system architecture is illustrated with respect to FIG. 9, and number of different architectures such as (but not limited to) those that do not include data centers, those that utilize different (or no) communication devices, and/or any other architecture capable of performing Spinal cord stimulator evaluation processes as described herein can be used as appropriate to the requirements of specific applications of embodiments of the invention.

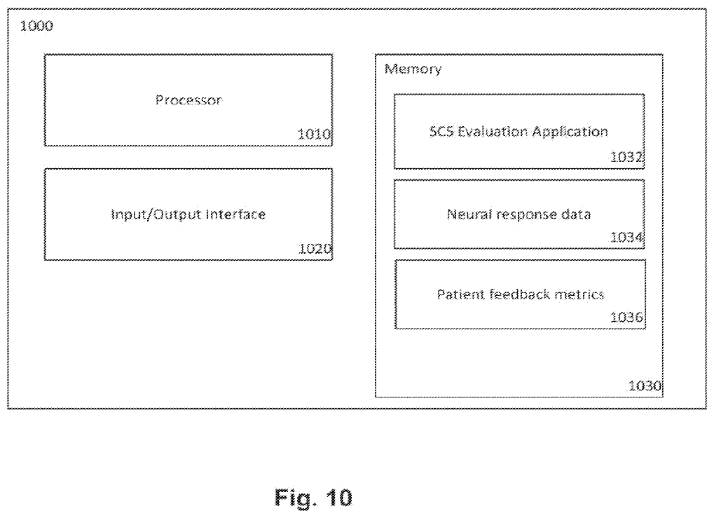

Turning now to FIG. 10, a block diagram for a SCS evaluator in accordance with an embodiment of the invention is illustrated. SCS evaluator 1000 includes a processor 1010. In many embodiments, the processor is a central processing unit (CPU), however any number of different logic processing circuitries (or combinations thereof) can be used, such as (but not limited to) graphics processing units (GPUs), field programmable gate arrays (FPGAs), application specific integrated circuits (ASICs), and/or any other logic processing circuit as appropriate to the requirements of specific applications of embodiments of the invention. The SCS evaluator 1000 further includes an input/output (I/O) interface 1020. I/O interfaces are capable of transmitting and receiving data from and to other devices, respectively. In numerous embodiments, multiple I/O interfaces may be included which operate using different communications modalities.

The SCS evaluator 1000 further includes a memory 1030. The memory can be made of volatile memory, non-volatile memory, and/or any combination thereof. The memory 1030 stores an SCS evaluation application 1032 which is capable of directing the processor to perform various spinal cord stimulator evaluation processes. In many embodiments, the memory 1030 stores neural response data 1034 obtained from a pulse generator, and patient feedback metrics 1036 at various points during operation.

While a specific SCS evaluator is illustrated in FIG. 10, as can readily be appreciated, any number of different computation architectures can be used as appropriate to the requirements of specific applications of embodiments of the invention. For example, in numerous embodiments, a hardware circuit designed to execute specific spinal cord stimulator evaluation processes can be used instead of general-purpose computing circuitry configured by an application without departing from the scope or spirit of the invention. Further, spinal cord stimulator evaluation processes are discussed in further detail below.

In numerous embodiments, electrodes are implanted into the patient and a pulse generator external to the body is used to provide diagnostic stimulation. Neural activity can be recorded by the electrodes and used to identify the presence and quality of ECAPs. Depending on the waveform of the ECAP, a confidence score can be calculated which indicates a degree of effectiveness of SCS on the patient. For example, the degree of effectiveness may be a likelihood that the SCS is effective to provide a sufficiently therapeutic benefit to the patient to justify implantation. In various embodiments, the confidence score is determined using one or more evaluation metrics which may include one or more functionality metrics and/or therapy quality metrics. Further, in various embodiments, postural effects are accounted for by evaluation metrics such as a postural robustness metric indicating the viability for the patient to have a stable therapeutic effect using closed loop stimulation. For example, the postural robustness metric indicates the viability for the patient's stimulation parameters to be automatically adjusted in closed-loop fashion without significantly impeding the quality of the SCS therapy.

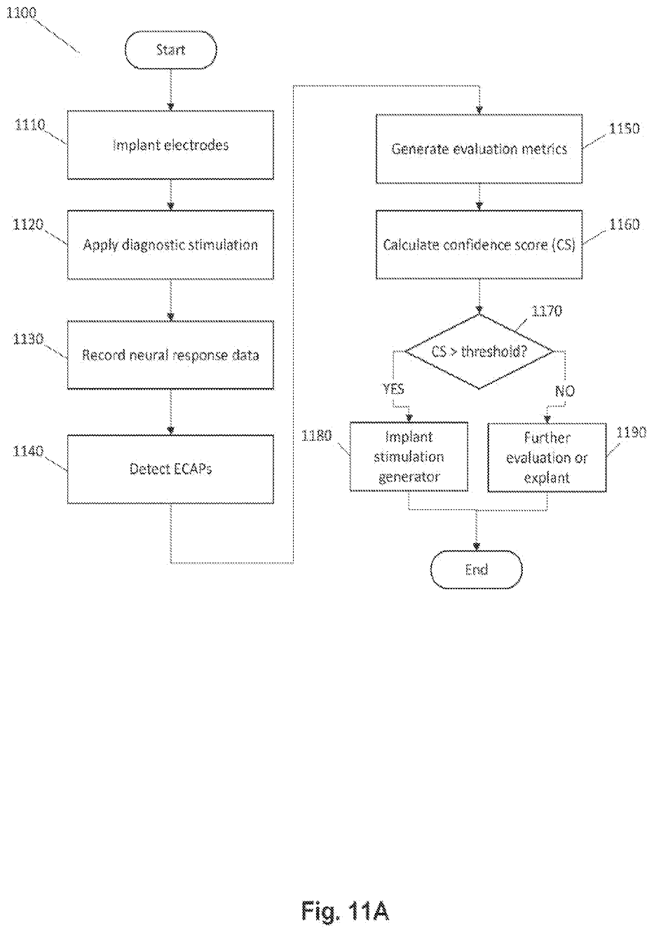

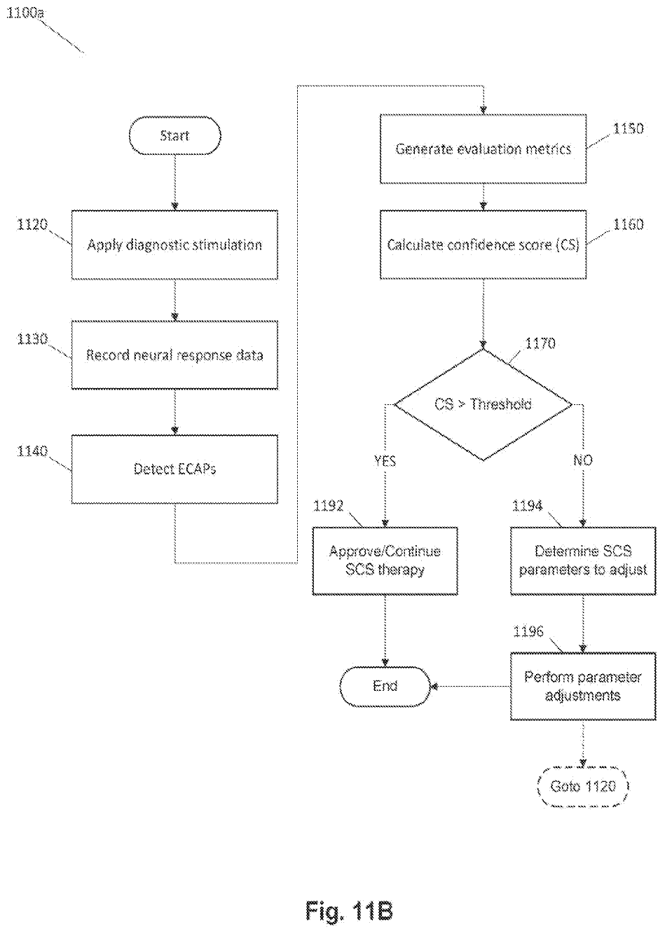

FIG. 11A illustrates a spinal cord stimulator evaluation process 1100 in accordance with an embodiment of the invention. Process 1100 includes implanting electrodes into the patient (1110). In numerous embodiments, the electrodes are surgically implanted into a patient's spinal cord. Diagnostic stimulation generated by a connected pulse generator is applied (1120) via the electrodes to the spinal cord. In numerous embodiments, the diagnostic stimulation is modified over time depending on the metrics being collected. In some embodiments, such as those directed to performing SCS evaluation in an SCS trial scenario, the diagnostic stimulation (also referred to as “trial” stimulation) is within a range to trigger awareness of effect by the individual patient without causing pain. In numerous embodiments, a virtual ground can be used in order to obtain a cleaner measurement of any ECAPs. Methodologies for using virtual grounds are discussed in further detail in U.S. Pat. No. 10,206,596, the disclosure of which is incorporated by reference in its entirety.

Following the application of the diagnostic stimulation, SCS evaluation according to process 1100 includes obtaining neural response data generated by a spinal cord stimulator. The neural response data describes one or more neural signals in the spinal cord of the patient, as recorded using the electrodes (1130). In numerous embodiments, neural response data includes a signal from each electrode configured to measure ECAPs. The signal indicates voltage of the cell membranes of the surrounding neurons over time. The neural response data can be timestamped and/or otherwise labelled such that the time to response of the neural tissue by a stimulation pulse generated by the pulse generator is known. In many embodiments, the neural response data is transmitted via the pulse generator to an SCS evaluator.

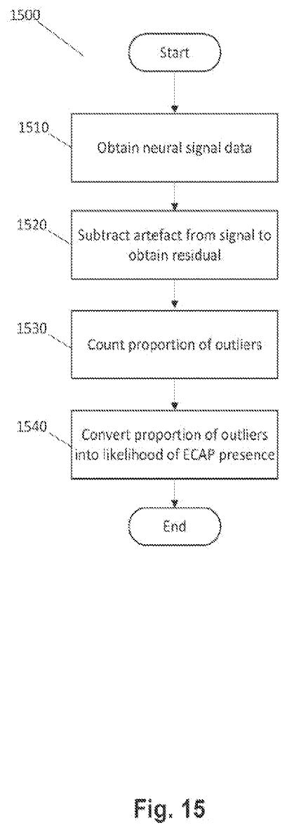

At step 1140, the process 1100 detects one or more evoked compound action potentials (ECAPs) associated with the one or more neural signals. One or more ECAPs are detected in the neural signal data, using a method such as shown in FIG. 15. In numerous embodiments, if an ECAP is not detected, additional diagnostic stimulation is applied at a higher current, and/or with modified stimulation parameters until an ECAP is detected. Once an ECAP is detected, the threshold current required to evoke a compound action potential is stored.

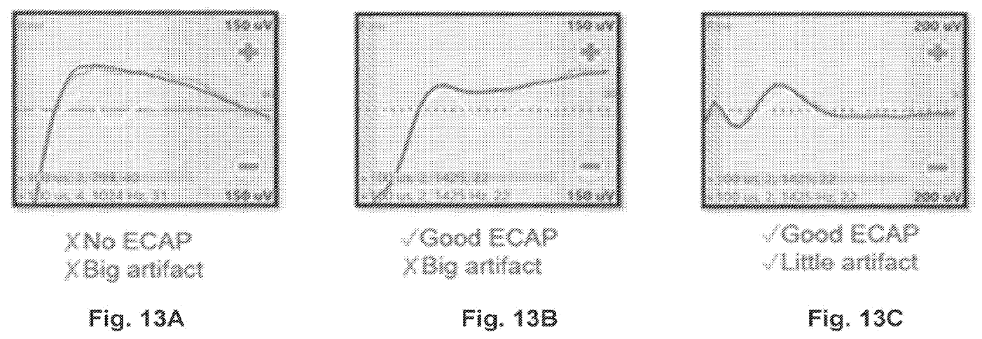

In numerous embodiments, the waveform of an ECAP can be highly indicative of a patient's response to SCS. In order to obtain a clear signal, artefact reduction techniques can be utilized such as (but not limited to) those described in International Patent Publication WO 2020/124135 titled “IMPROVED DETECTION OF ACTION POTENTIALS” filed Dec. 17, 2019, the entirety of which is hereby incorporated by reference. Examples of an ECAP signal with little artefact in accordance with an embodiment of the invention is illustrated in FIG. 13C. For comparison, an ECAP signal with significant artefact in accordance with an embodiment of the invention is illustrated in FIG. 13B; and a signal which illustrates a lack of an ECAP in response to stimulation with significant artefact in accordance with an embodiment of the invention is illustrated in FIG. 13A.

At step 1150, the process 1100 processes the one or more detected ECAPs to generate one or more evaluation metrics. The evaluation metrics may include one or more of a number of different patient functionality metrics that are derived from the properties of the detected ECAP signal, including (but not limited to) an ECAP signal to noise ratio (SNR), an ECAP signal to artefact ratio (SAR), an ECAP likelihood indicating the confidence with which the given ECAP is detected, morphological stability metrics, signal to artefact ratio coefficient of variance (SAR CoV), a signal quality indicator, activation plot quality metrics, and/or any other patient functionality metric derived from ECAPs as appropriate to the requirements of specific applications of embodiments of the invention.

In some embodiments, additional patient functionality metrics are obtained from sources outside of ECAP signals. For example, in various embodiments, the additional patient functionality metrics include (but are not limited to) dermatome activation, visual analog scale (VAS) score, heart rate variability in response to stimulation, pupil dilation in response to stimulation, gait regularity, and/or performance on any other functionality test as appropriate to the requirements of specific applications of embodiments of the invention. In many embodiments, these measurements can be obtained near immediately within a standard hospital setting. The evaluation metrics may include, either in addition, or as an alternative, to the functionality metrics, one or more therapy quality metrics, as described below.

At step 1160, the one or more evaluation metrics are used to generate a confidence score indicating a degree of effectiveness of SCS on the patient. FIG. 11A is directed to a SCS trial scenario in which the confidence score indicates the likelihood that a patient will receive sufficient therapeutic benefit from SCS to justify implantation.

In some embodiments, the confidence score is calculated using one or more machine learning models trained using supervised training with values of evaluation metrics obtained from a training data set. The training data values are annotated with respective indications of whether or not a therapeutic benefit from SCS was achieved for one or more sample patients. In various embodiments, the annotations indicate whether or not a clinician recommended SCS for the sample patient. In some embodiments, the machine learning model is trained using ECAP parameter and/or evaluation metric values labelled with respective patient outcomes. Each machine learning model may be any of a number of different machine learning models including (but not limited to) a neural network, a support-vector machine, a regression model, and/or any other supervised machine learning model capable of outputting a probability as described herein.

In some embodiments, the confidence score is determined as a sum of the values of the evaluation metrics, such as for example a weighted sum of the values of one or more functionality metrics, therapy quality metrics, and/or any other metrics derived from or related to the detected ECAPs.

FIG. 11A illustrates an exemplary approach to using the generated confidence score to determine a likelihood that the SCS is effective to provide a therapeutic benefit to the patient. At step 1170, the confidence score is compared to a predetermined threshold value. In response to the confidence score exceeding the predetermined threshold value, the patient is deemed likely to significantly benefit from SCS, and the process 1100 provides a positive indication for implantation of the stimulator (1180).

In response to the confidence score failing to exceed a predetermined threshold value, the patient is deemed unlikely to significantly benefit from SCS. In this case, at step 1190, an indication may be provided of one or more additional activities to further assess whether the SCS is likely to provide a long-term therapeutic effect to the patient. For example, the SCS evaluator may provide recommendations for additional trial procedures, which may include further SCS evaluation by repeating steps 1120-1170. Alternatively, the SCS evaluator may provide an instruction or recommendation for explantation of the spinal cord stimulator, and/or one or more leads, from the patient.

In some embodiments, a separate success threshold and failure threshold are used to determine whether the SCS is likely to be effective to provide a therapeutic benefit to the patient. For example, the generation of a confidence score that exceeds the success threshold may be interpreted by the SCS evaluator to indicate that SCS is likely to have significant therapeutic benefit for the patient, while the generation of a confidence score below the failure threshold may be interpreted by the SCS evaluator to indicate that SCS is unlikely to significantly benefit the patient. In some embodiments, in response to the generation of a confidence score value between the failure threshold and the success threshold, the SCS evaluator is configured to recommend referring the patient to a medical professional and/or an extension of the implantation trial period. In this way, the complete spinal cord stimulation implantation procedure can be performed within 24 hours, removing the need for multiday or multiweek trial periods.

FIG. 11B illustrates a second SCS evaluation process 1100a in accordance with an embodiment of the invention. In process 1100a, the confidence score is used to perform a viability assessment of a SCS program, and/or to adjust parameters of the SCS program to provide improved therapeutic benefit for the patient. Implantation of the electrodes into the patient is performed prior to the development and assessment of an SCS program, resulting in the omission of step 1110 (compared to process 1100 of FIG. 11A).

At step 1120, diagnostic stimulation is applied by a pulse generator connected to the implanted electrodes. The diagnostic stimulation is applied in accordance with the SCS program being evaluated by the SCS evaluator. For example, the diagnostic stimulation may involve the application of a stimulus signal where the properties of the stimulus signal, and/or its application, are selected to replicate the actual or intended effect of the SCS program.

One or more SCS program parameters may be used to determine corresponding properties of the diagnostic simulation. In the described embodiments, SCS program parameters include but are not limited to: one or more parameters specifying a stimulus signal (e.g., one or more signal intensities, and at least one signal frequency); and one or more parameters specifying a schedule determining the application of the stimulus signal to stimulate the spinal cord of the patient (e.g., time values indicating a stimulus periodicity, and total duration of the therapy). In some embodiments, the SCS program parameters include one or more electrode configuration parameters specifying operational characteristics of the electrodes of the stimulator. In some embodiments, the SCS program parameters include an SCS program mode indicating whether the stimulator operates in an open-loop mode or a closed-loop mode to apply the stimulus signal. In the case of closed-loop operation, the SCS program parameters may also include an indication of a neural recruitment target level used by the stimulator.

Steps 1130 to 1160 are performed, by the SCS evaluator, as described above for process 1100 of FIG. 11A. At step 1170, the SCS evaluator compares the confidence score calculated at step 1160 to a predetermined threshold value. In some embodiments, the predetermined threshold value indicates a minimum level of confidence of an SCS program that is expected to provide a significant beneficial therapeutic effect to the patient. In response to the confidence score exceeding the predetermined threshold value, at step 1192 the SCS evaluator determines an approval of the SCS therapy for treating the patient. In some embodiments, the SCS evaluator is configured to provide an indication to a user of the system or device (e.g., a clinician) that the SCS program should be prescribed to the patient (i.e., where the SCS program comprises or is part of a proposed treatment), or that the SCS program should continue to be applied to treat the patient (i.e., where the SCS program comprises or is part of a present treatment).

In response to the confidence score failing to exceed the predetermined threshold value, the SCS evaluator is configured to determine one or more parameters of the SCS program for adjustment (i.e., at step 1194). The SCS program parameters selected for adjustment may vary depending on the one or more evaluation metrics used to calculate the confidence score, the degree to which the confidence score failed to exceed the predetermined threshold, and/or any the values of the one or more evaluation metrics generated in response to the diagnostic stimulation (as described below in relation to the therapy quality metrics).

At step 1196, the SCS evaluator performs one or more parameter adjustment operations to adjust the determined one or more SCS parameters. In some embodiments, the one or more SCS parameters are adjusted by an amount determined, at least in part, by processing the one or more evaluation metric values. Optionally, the SCS evaluator is configured to re-evaluate the SCS program following the adjustments to the program parameters by recommencing step 1120 to apply new diagnostic stimulation to the patient. The new diagnostic stimulation is applied in accordance with the adjusted SCS program parameters. In some embodiments, the SCS evaluator is configured to iteratively repeat the application of diagnostic stimulation associated with an SCS program (step 1120), the evaluation of the program (steps 1130-1170), and the adjustment of program parameters (step 1196) until the SCS program provides a suitably effective therapeutic benefit for the patient.

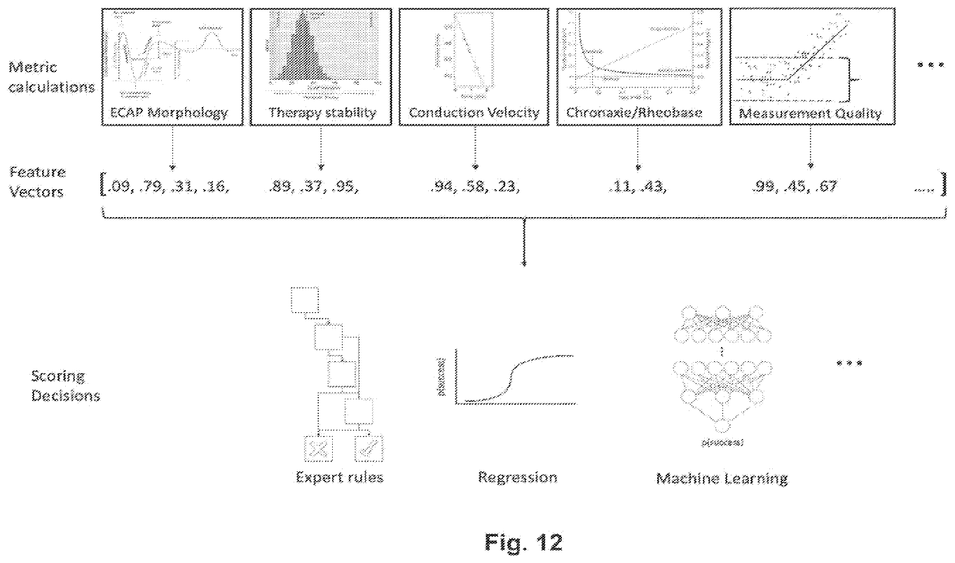

As can be readily appreciated, while specific processes for evaluating spinal cord stimulation are illustrated by FIG. 11A and FIG. 11B, any number of different processes can be used without departing from the scope of the invention. An example process for evaluating spinal cord stimulator implantation in accordance with an embodiment of the invention is illustrated in FIG. 12. As shown in FIG. 12, multiple different methods can be used to calculate a confidence score. In various embodiments, one or more methods are used to calculate a confidence score. Multiple confidence scores can be averaged to create an ensemble confidence score which can be used in place of a single confidence score.

In some embodiments, multiple confidence scores are compared to multiple corresponding threshold values. The SCS evaluator provides an SCS evaluation outcome (i.e., an indication of SCS program viability, or whether the patient is likely to significantly benefit from SCS therapy) based on whether some, a majority, or all confidence scores exceed the respective threshold values. For example, an indication that the patient would benefit from SCS may require all confidence scores to exceed their respective threshold values. In one exemplary implementation, a machine learning model is provided with at least values of evaluation metrics discussed herein and, in response, provides a classification of SCS program viability or general SCS efficacy for the patient, rather than a confidence score. In some embodiments, additional measurements can be used to inform the calculation of the confidence score. Processes for calculating evaluation metrics that are used to determine the confidence score(s) are described in further detail below.

### Patient Functionality Metrics

Patient functionality metrics (also referred to as just “functionality metrics”) are metrics obtained via observation of the patient, measurement of biological signals, and/or self-reporting by the patient. While some patient functionality metrics can be recorded using industry standard practices (e.g. gait via motion detection cameras, heart rate variability via heartrate monitor, pupil dilation via camera, pain questionnaires), others are unconventional and are described below.

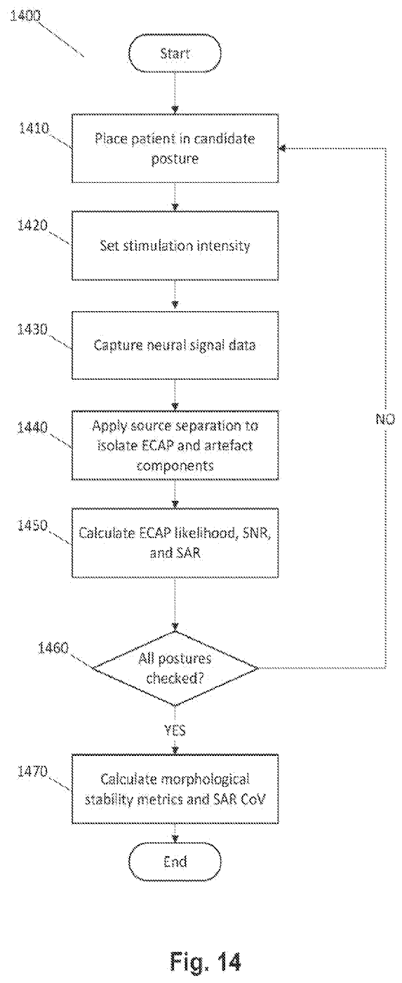

The ECAP can provide significant information regarding the viability of SCS for a particular patient. Further, the ECAP can be characterized to provide additional predictive power. In numerous embodiments, collecting many ECAP measurements during which the patient is in a number of different postures can give an even better prediction of SCS viability for a given patient. Turning now to FIG. 14, a flow chart for deriving certain patient functionality metrics from neural signal data possibly containing ECAPs in accordance with an embodiment of the invention is illustrated.

Process 1400 includes placing (1410) the patient in a candidate posture. In numerous embodiments, the candidate posture is at least one of standing, sitting, lying down, prone, lying on side with back straight, lying on side with back arched, and/or any other position that the patient might commonly find themselves in. Stimulation intensity is set (1420) to a comfortable and therapeutic level. In many embodiments, the stimulation intensity is set at a level reported as comfortable by the patient. In various embodiments, the stimulation intensity is set at a small multiple of the minimum current required to trigger an ECAP (the ECAP threshold). In some embodiments, the stimulation intensity is set at a multiple and/or scaling factor of where the ECAP presence is detected.

A stimulus is delivered at the stimulation intensity and neural signal data is captured (1430). A source separation algorithm is applied (1440) to isolate the ECAP component and any artefact components from the neural signal. An ECAP likelihood, signal-to-noise ratio (SNR), and signal-to-artefact ratio (SAR) are calculated (1450) from the isolated components. Specific methods for calculating ECAP likelihood, SNR, and SAR are discussed in subsections below. If neural signal data has not (1460) been captured from all candidate postures, then the patient is placed in the next candidate posture position and the above is performed again. Once all candidate postures have (1460) been checked, the separated components acquired from the different candidate postures are used to calculate (1470) morphological stability and SAR coefficients of variance (SAR CoV).

As can be readily appreciated, other metrics can be calculated without departing from the scope or spirit of the invention. Further, the number and type of candidate postures can vary depending on the particular patient scenario as appropriate to the requirements of specific applications of embodiments of the invention. Specific calculations for the above metrics are described below.

### ECAP Likelihood

ECAP likelihood is a measurement of the probability that a neural signal contains an ECAP. In some embodiments, an ECAP detector can provide the ECAP likelihood instead of the evaluator. As noted above with respect to FIGS. 13A-C, ECAPs have a particular basic morphology. Turning now to FIG. 15, a process 1500 for calculating ECAP likelihood in accordance with an embodiment of the invention is illustrated. Once the artefact component is obtained (1510), the artefact component is subtracted (1520) from the neural signal to obtain a residual. The proportion of outliers are counted (1530). In numerous embodiments, a residual outlier is a value outside of +Nσ, where σ is the standard deviation of the measurement chain noise. The proportion of outliers are converted (1540) into a likelihood of ECAP presence. In many embodiments, the ECAP likelihood is a value between [0, 1], where 1 means an ECAP is present, and 0 means the signal is indistinguishable from measurement chain noise.

While a particular process for calculating ECAP likelihood is described in FIG. 15, ECAP likelihood can be calculated in other ways as appropriate to the requirements of specific applications of embodiments of the invention. For example, in various embodiments, a machine learning classifier can be trained to locate ECAPs in neural signal data and provide a likelihood without departing from the scope or spirit of the invention. ECAP likelihood can be used for numerous purposes such as SCS viability prediction. In various embodiments, ECAP likelihood can be used to judge the quality/trustworthiness of SNR and SAR measurements.

### ECAP SNR and SAR

Signal-to-noise ratio and signal-to-artefact ratio are important metrics which can be derived from neural signal data. In numerous embodiments, high SNR and SAR equate to better control of neural recruitment, and are therefore desirable for successful outcomes. Specifically, SAR stability across multiple postures is often desirable for more precise recruitment control.

SNR and SAR can be calculated in similar ways using the ECAP and artefact components obtained via source separation of the neural signal (1440). In numerous embodiments, SNR is calculated by subtracting the artefact and ECAP components from the neural signal to obtain a residual, and subsequently calculating SNR as:

$$S N R = \frac{{V}_{rms} ( EeCAP )}{{V}_{rms} ( residual )}$$

Similarly, the SAR can be calculated as:

$$S A R = \frac{{V}_{rms} ( EeCAP )}{{V}_{rms} ( artefact )}$$

### ECAP Morphological Stability

ECAP morphological stability is a measurement of how similar the ECAP waveform is across multiple instances and/or postures. ECAP morphological stability, like SAR stability across multiple postures, is often desirable. Having a high degree of morphological stability can indicate a higher chance of success of SCS implantation. In numerous embodiments, ECAP morphological stability is calculated using neural signal data of ECAPs recorded during different candidate postures. For each source separation of the recording, morphological parameters can be derived as a function of posture. Once the morphological parameters have been derived, a coefficient of variation for each morphological parameter can be computed. In some embodiments, the various coefficients of variation are synthesized into a single number, such as discussed in PCT Publication WO2021/007615A1 titled “Monitoring a quality of neural recordings” filed Jul. 13, 2020, the disclosure of which is hereby incorporated by reference in its entirety. In various embodiments, the coefficients of variation are instead provided to a machine learning model as distinct numbers (either individually and/or via a data structure containing multiple coefficients).

### Activation Plot Quality Metrics

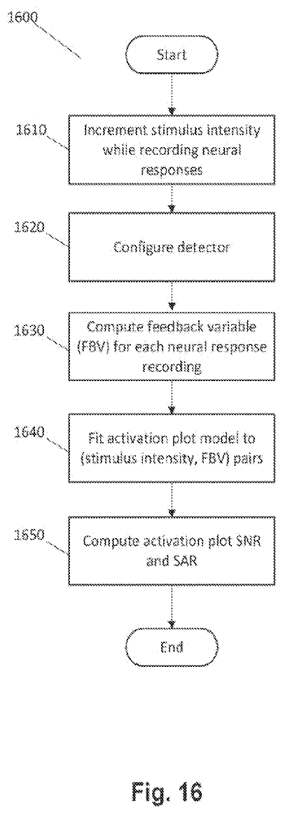

As discussed above, an activation plot, or growth curve, is an approximation to the relationship between stimulus intensity (e.g. an amplitude of the stimulus current pulse) and intensity of neural response resulting from the stimulus (e.g. an ECAP amplitude). Activation plots can further be provided to the machine learning model to predict SCS viability for a patient. Turning now to FIG. 16, a process 1600 for computing activation plot quality metrics in accordance with an embodiment of the invention is illustrated.

Process 1600 includes incrementing (1610) stimulus intensity to the patient while recording neural responses. In numerous embodiments, the stimulus intensity is increased to a multiple of the recruitment (ECAP) threshold level. In various embodiments, the intensity is increased up to between 1.3× and 1.6× the threshold. In many embodiments, an ECAP detector is configured (1620) to account for the ECAP shape and duration and offset from the stimulus pulse. Configuration of ECAP detectors is discussed in U.S. Pat. No. 10,426,409 titled “Method and device for detecting a neural response in a neural measurement” filed Nov. 22, 2013, the disclosure of which is hereby incorporated by reference in its entirety. This can be repeated while the patient is in a number of different candidate postures to obtain multiple neural responses. A neural recruitment magnitude (NRM) for each neural response recording is computed (1630). In numerous embodiments, the feedback value is the amplitude of the measured ECAP. The activation plot model is fitted (1640) to (stimulus intensity, NRM) pairs, and activation plot quality metrics such as SNR and SAR are computed (1650). In numerous embodiments, SNR and SAR for each activation plot are computed similarly as described above with respect to the ECAP. A discussion of activation plot SNR can be found in International Patent Publication No. WO 2021/007615 titled “MONITORING A QUALITY OF NEURAL RECORDINGS” filed Jul. 13, 2020, the disclosure of which is incorporated by reference in its entirety.

### Postural Robustness Metric

In some embodiments, the SCS evaluator is configured to utilize a postural robustness metric as one of the evaluation metrics used to generate the confidence score. The postural robustness metric indicates the viability for the patient to have a generally stable therapeutic effect from SCS by measuring the robustness of NRMs to postural changes. In many embodiments, spinal cord stimulators are capable of closed loop operation. Closed loop operation refers to the automatic tuning of stimulation parameters in response to changes in posture and other movements. For example, as a patient moves their body, due to changing pressures in the spine, stimulation at different electrodes may need to be modified to maintain comfortable operation. In a closed loop system, these changes are automatically made in response to detected changes. In contrast, an open loop system refers to one where the patient or doctor must manually tune the stimulation using a controller.

The calculation of the postural robustness metric may be performed with the SCS program mode set to either open-loop or closed-loop control. Comparing the postural robustness metric values for a patient across the SCS program modes may provide an indication of the relative benefit of closed loop SCS for the patient.

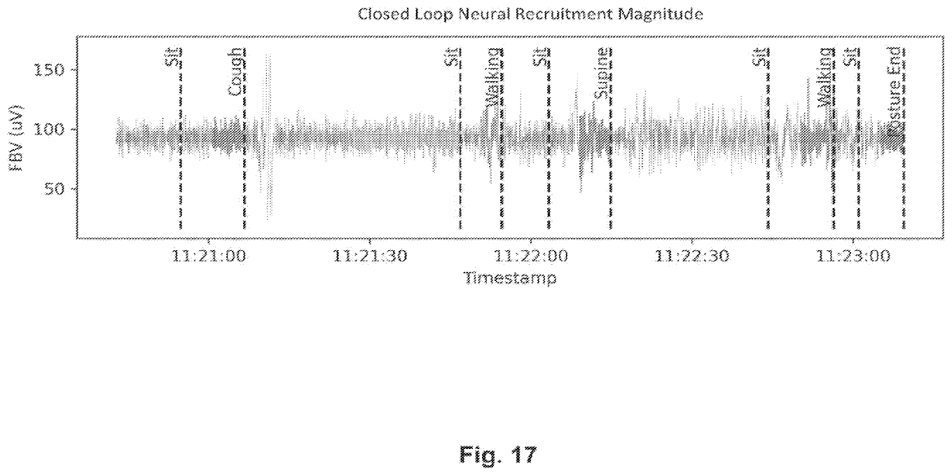

In some embodiments, the NRMs described above can be used to determine the programming of closed loop stimulation. NRMs are calculated multiple times as a patient works through a set of postures, e.g. sitting, standing, walking, bending over, twisting, rolling over, couching, supine, etc. Example changes in NRM variability in a typical scenario during different positions in accordance with an embodiment of the invention are illustrated in FIG. 17. The changes in NRM variability for each posture can be determined and used to calculate the postural robustness metric. The postural robustness metric may be used to assess the viability of either open-loop or closed-loop control modes. For example, in response to the postural robustness metric exceeding a given threshold in closed-loop operation, the individual NRM values can be used to program a closed loop system.

In many embodiments, when keeping the loop gain, G, constant, the noise on the NRM changes with patient sensitivity. Patient sensitivity can change with posture, and the size of the neural recording increases as electrodes get closer to the spinal cord. Change in sensitivity can be computed by measuring the standard deviations of the NRM in different postures. In various embodiments, closed loop stimulation works better for patients that show less variation in sensitivity with posture than patients who exhibit a lot of variation relative to some arbitrary baseline posture. Therefore, postural variation of sensitivity can be quantified by measuring the CoV of the NRM standard deviation in different postures. In numerous embodiments, this is simpler than generating multiple activation plots because there is no need to manually adjust the current or worry about over stimulating the patient during testing.

Postural variations in SCS are accounted for by recording, in response to the SCS, a set of NRMs of the detected ECAPs when the patient is in multiple candidate postures. In the described embodiments, determining the postural robustness metric comprises: (i) providing SCS to the patient via the spinal cord stimulator, when the patient is positioned in a candidate posture: (ii) recording, in response to the SCS, a set of neural recruitment magnitudes (NRMs) of the detected ECAPs associated with the neural signals in the spinal cord of the patient in the candidate posture; and (iii) determining a first measure of variation to measure variation in the values of the set of NRMs. For example, the first measure of variation may be determined by calculating a standard deviation value of the set of NRMs obtained from the patient in the candidate posture (i.e., to provide an indication of the intra-set variation in NRM values). As an additional step, a second measure of variation is computed of a plurality of first measures of variation (e.g., standard deviation values), wherein the plurality of first measures of variation are obtained by iteratively performing steps (i)-(iii) for different candidate postures.

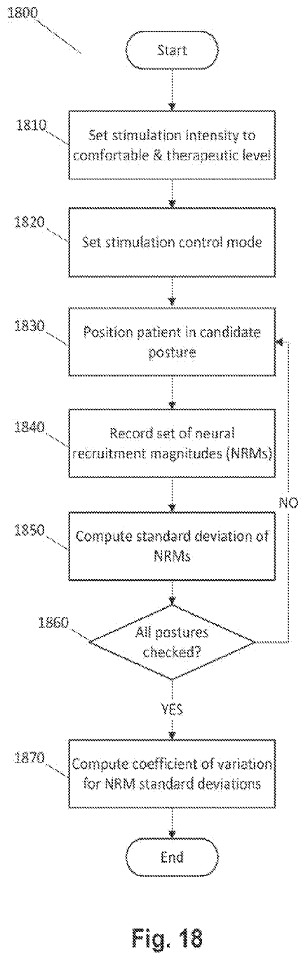

FIG. 18 illustrates an exemplary process 1800 for evaluating postural robustness of SCS in accordance with an embodiment of the invention. Process 1800 includes first setting (1810) stimulation intensity to a comfortable and therapeutic level for the patient. The stimulation control mode is set to closed loop control in the described example (i.e., at 1820). However, in other evaluations the control mode may be set to the open-loop mode. The patient is placed (1830) in the first candidate posture. NRMs are recorded (1840) and the standard deviation of the recorded NRMs is computed (1850)). This process is repeated (1860) for all candidate postures. The coefficients of variation for the NRM standard deviations are computed (1870). In some embodiments, the coefficients of variation for the NRM standard deviations are used to program closed loop operation of the stimulator. For example, the coefficients of variation for the NRM standard deviations may be processed (e.g., by comparison to one or more predetermined thresholds) to provide a clinician or other user with information as to the postural variation or sensitivity of the SCS for the patient. The clinician or other user may subsequently set or adjust one or more parameters of an SCS program to account for the postural variation, thereby improving the effectiveness of the SCS therapy for the patient.

### Therapy Quality Metrics

The functionality metrics and postural robustness metrics described above can generate confidence measures that indicate SCS efficacy based on the properties of the ECAPs detected from the applied stimulus. In some embodiments, functionality and postural robustness metrics are supplemented with therapy quality metrics that provide an indication of SCS effectiveness according to an actual, or expected/proposed, therapeutic use case of the patient. While clinical assessments of SCS therapy effectiveness may include subjective factors (e.g., the mood, motor function, pain perception, sleep quality, and general quality of life of the patient), the therapy quality metrics described herein provide an objective measure of SCS efficacy by quantifying error, accuracy, or variability of parameters associated with the proposed or actual prescribed therapy.

Some of the therapy quality metrics described below quantify error, accuracy, or variability of the detected neural response of the patient relative to one or more corresponding baseline target therapy levels. Variability relative to the baseline target level(s) may be calculated using one or more statistical measures such as, for example, root mean square error (RMSE), absolute error (sum of absolute deviations, median of absolute deviations, mean of absolute deviations, etc.), range of observed values (interquartile range, percentile ranges, e.g. 10th to 90th percentile), standard deviation, and/or any other calculation as appropriate to the requirements of specific applications of embodiments of the invention. In a variety of embodiments, the baseline is determined at the start of an evaluation, although the baseline can be a prescribed in-clinic therapy level, or a stimulation target level when the device is used out-of-clinic.

In the described embodiments, determining values of a therapy quality metric that quantifies the error, accuracy, or variability of the detected neural response includes: (i) providing SCS to the patient (i.e., via the spinal cord stimulator) when the patient is positioned in a candidate posture: (ii) recording, in response to the SCS, a set of NRMs of the detected ECAPs associated with the neural signals in the spinal cord of the patient in the candidate posture; and (iii) processing the recorded set of NRMs to compute values of the one or more therapy quality metrics. In some embodiments, the therapy quality metrics also account for postural effects on the patient by determining the metric values across different postures, and/or individually for data collected for a specific posture or movement, and then aggregating the values. For example, in one implementation the steps (i)-(ii) above are iteratively repeated for different candidate postures such that step (iii) includes processing respective sets of NRMs recorded for the different candidate postures.

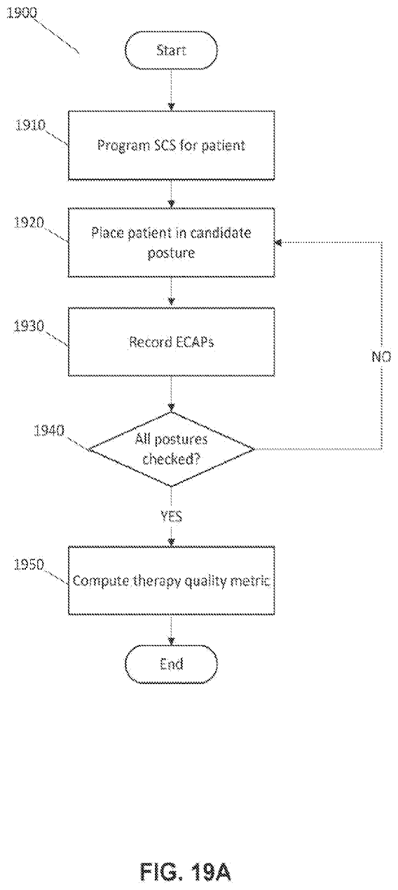

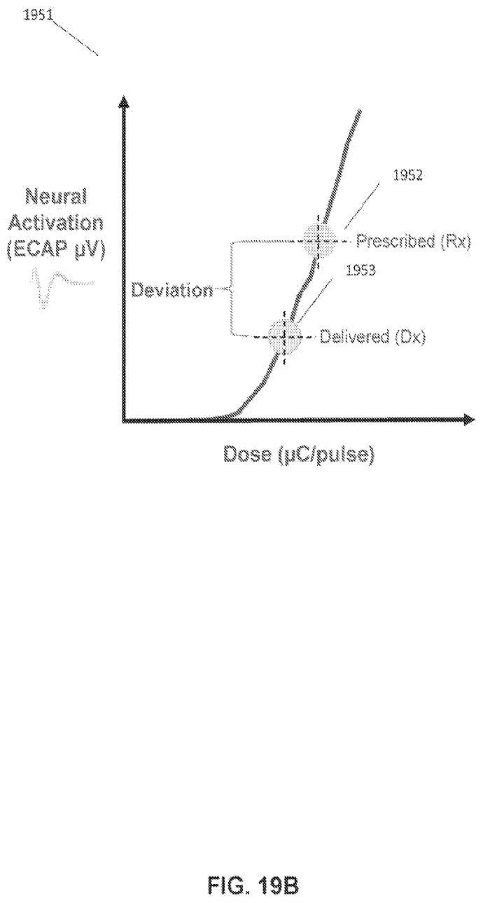

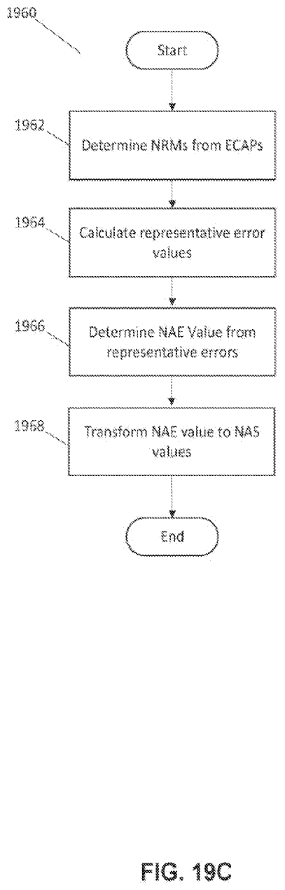

FIG. 19A illustrates an exemplary process 1900 for calculating therapy quality metrics in accordance with an embodiment of the invention. As an optional initial step 1910, the SCS for the patient is programmed (i.e., by setting one or more initial values of the SCS program parameters). In numerous embodiments, the SCS program mode is set to the closed-loop mode. However, this process can equally be performed using an open-loop programming regime. The patient is placed (1920) in a candidate posture and ECAPs are recorded (1930). If not all postures have been checked (1940), then the patient is placed in the next candidate posture (1920) and more ECAPs are recorded (1930). In many embodiments, the patient is instructed to move into candidate postures (or movements) that are most like their every-day movements. In various embodiments, instead of candidate postures, ECAPs are recorded during the patient's day/night as they move normally. Once all ECAPs have been recorded, the one or more therapy quality metrics are computed (1950).

### Therapy Utilization and Therapy Target Level

In some configurations, the one or more therapy quality metrics are used to generate the confidence score, either alone or in combination with one or more functionality metrics and/or postural robustness metrics. The therapy quality metrics described herein may be in a raw form or normalized to any relevant patient therapy target levels or ranges.

Exemplary therapy quality metrics include metrics related to the usage of the SCS therapy program by the patient, including: a therapy utilization value; and/or a therapy target level or target level value.

The therapy target level may be a value of a comfort level, maximum level, a minimum level, or any target level of the SCS program for delivering a prescribed or actual SCS therapy to the patient. The therapy target level value indicates a difference between one or more observed therapy target levels and the prescribed therapy target level, or another specified level or threshold (e.g. stimulation perception threshold, objective ECAP threshold, subjective Maximum stimulation level, or an objectively predicted Maximum stimulation level such as 1.4× ECAP Threshold).

Metrics related to the therapy target level therefore provide an objective assessment of the actual target level of activation of the therapy, and/or a degree of variation between the actual target level and a target level prescribed to the patient (i.e., to assess what changes the patient has made to the prescribed target level in their actual therapeutic use case). For example, the SCS evaluator may be configured to calculate an average of the observed therapy target levels (as set by the patient) over a predetermined window to determine a time-varying function of the target therapy level differential value. Reductions in the values of the differential function over time may indicate an SCS program that is more closely aligned with patient feedback, thereby resulting in a higher confidence score.

The therapy utilization value is a measure of how much and/or how often the patient uses the SCS therapy. In one configuration, the therapy utilization value may be determined as a ratio of the average number of minutes for which SCS therapy is performed on the patient compared to a base value. For example, the therapy utilization value may indicate a daily average of the time, either as a raw value or a percentage of elapsed time, that the SCS therapy is active for the patient. In another configuration, the therapy utilization value may be determined as a fraction of therapy time during which the stimulus intensity is above the ECAP threshold.

In some embodiments, additional therapy quality metrics may be included within the one or more evaluation metrics, such as, for example, a subjective Threshold to Maximum therapeutic range. Such a range may be specified in terms of neural activation (e.g. NRM in uV, or peak or peak-to-peak amplitudes) or stimulation current (mA); and/or an objectively predicted therapeutic range.

### Neural Activation Accuracy Measures

In some embodiments, the therapy quality metrics include one or more measures related to the neural activation accuracy of the evaluated SCS therapy. Neural activation accuracy measures provide an objective assessment of how consistently and effectively neural activation is achieved in an actual therapeutic use case by the patient.

FIG. 19B illustrates a graph (1951) of a measure of neural activation accuracy based on a deviation between the ECAP activation achieved by prescribed (1952) and delivered (1953) doses of a neural stimulus. A high degree of deviation between prescribed and delivered neural activation over time is indicative of a low level of neural activation accuracy (i.e., since the delivered level of activation often is significantly above or below the target activation level). Conversely, a low degree of neural activation deviation is indicative of a high level of neural activation accuracy (i.e., since the delivered level of activation often closely approximates the target activation level).

Neural Activation Error (NAE) is an objective measure of neural activation accuracy based on a quantification of the relative deviation in measured neural recruitment values (i.e., the NRMs of the detected ECAP response) from a baseline value of a therapy level. In the described embodiments, the NAE values are defined by calculating the root mean squared error (RMSE) of the NRM values as:

$$N A E [ μ V ] = \sqrt{\frac{{\sum }_{i = 0}^{n} {( N R {M}_{i} − N R {M}_{base \_ i} )}^{2}}{n}}$$

where NRMᵢ is the i-th sample of a neural response magnitude obtained from the patient (which may be specific to a particular posture), and NRM_{base_i} is an average NRM measured across a baseline period. In various embodiments, NRM_{base_i} is constant for i=0 to N. In numerous embodiments, NRM_{base_i} is the instantaneous therapy target level (for example a closed loop NRM target level for an individual sample i).

In a variety of embodiments, the NAE values are transformed to generate a therapy quality metric in the form of a Neural Activation Accuracy Score (NAS). Use of the NAS as a therapy quality metric is advantageous in enabling a standardized assessment of neural accuracy for an SCS program. That is, the representation of neural activation accuracy by the NAS values facilitates neural activation accuracy comparisons across patients for given evaluated SCS therapies, and assists clinicians with interpretation of the values.

FIG. 19C illustrates a process 1960 for determining values of the NAS in accordance with an embodiment of the invention. First, as described above, one or more sets of NRMs are recorded from the detected ECAPs associated with the neural signals invoked by providing SCS to the patient via the spinal cord stimulator (1962). A representative error value of each recorded set of NRMs is calculated relative to corresponding NRMs of a baseline target therapy level (1964). In some examples, the representative error value of the set of NRMs is a RMSE value calculated as shown above.

A neural activation error (NAE) value is determined (1966) by processing one or more of the calculated representative error values. For example, the NAE value may be determined as the RMSE of the posture dependent sets of NRMs, or as a statistical measure derived from the same. The NAE value is then transformed to generate a value of the NAS between zero and a predetermined maximum value (1968).

In some embodiments, the NAE values are transformed by applying one or more normalization or scaling operations to calculate the NAS values. For example, the NAS may be calculated by normalizing the NAE with reference to a baseline feedback variable value (FBV_{base}):

$$N A S = \frac{N A E}{{FBV}_{base}}$$

$$or$$

$$N A S = N A E - {FBV}_{base}$$

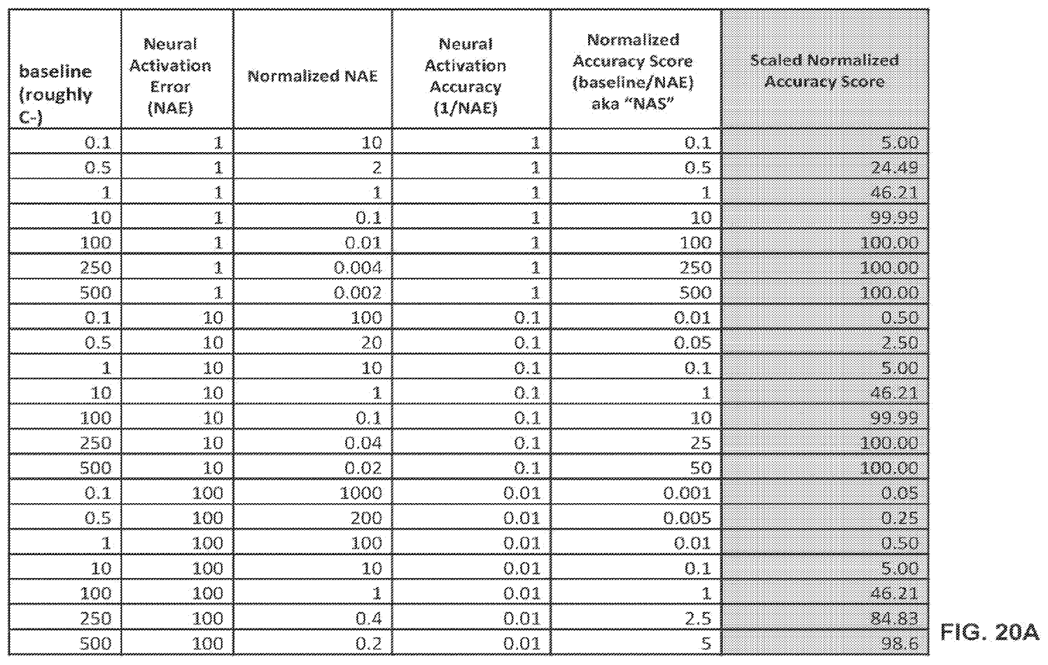

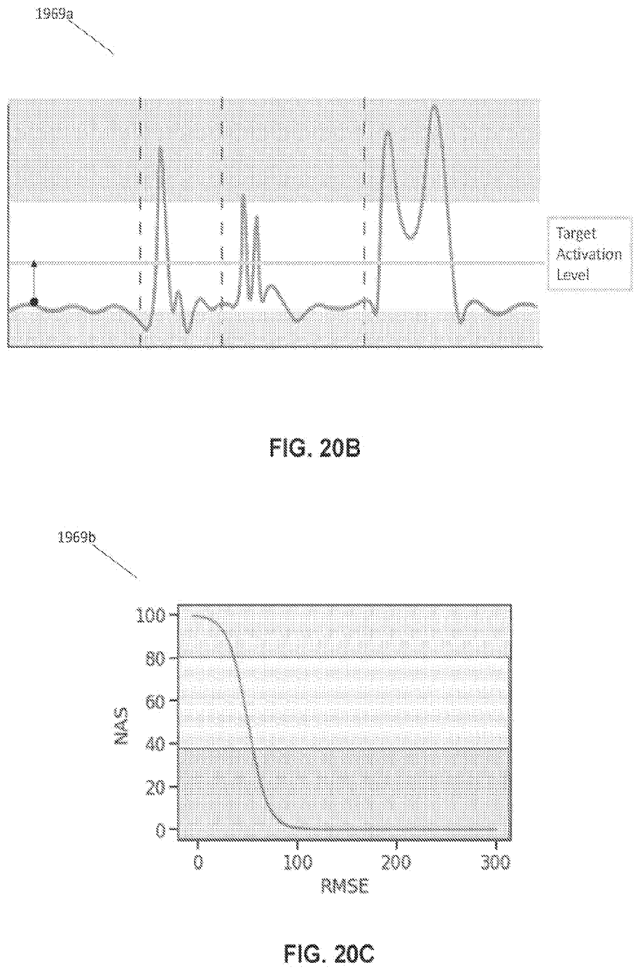

In various embodiments, the SCS evaluator is configured to generate values of an inverse of the respective NAE values. As illustrated, many metrics related to the NAE may be generated during, or due to, transforming the NAE to produce the NAS, including (but not limited to) 1/NAE and baseline and/or target therapy levels or ranges divided by NAE. In numerous embodiments, error metrics (RMSE, or other) are scaled into a fixed range to be more readily clinically useful and/or so that they can be associated with thresholds for clinical decisions. This is illustrated in the chart containing arbitrary example data presented in FIG. 20A in accordance with an embodiment of the invention.

Normalization of the NAE values is advantageous in contextualizing the impact of error within a patient's therapy level. For example, a 10 μV change may be more significant when the therapy target is 20 μV compared to 100 μV. In some embodiments, the scaling and/or normalization applied to the NAE values results in the generation of NAS values within the range of 0 (poor) to 100 (perfect), as shown in the right column of the chart in FIG. 20A.

In some embodiments, the NAS values are generated by transforming the deviations or errors in the feedback variable values according to a transformation function. For example, NAS values may be calculated by: (1) determining a point-by-point deviation in the measured and target FBV values, such as by subtracting the target from the measured FBV value: (2) calculating a RMSE value of the point-by-point deviation according to

$$R M S E = \sqrt{\frac{{\sum }_{i = 0}^{n} {( {FBV}_{m easured \_ i}^{n} − {FBV}_{target \_ i} )}^{2}}{n}}$$

and (3) inputting the RMSE into a sigmoid function to transform the values to real numbers within the interval of 0-100, according to

$$N A S = \frac{100}{1 + {e}^{m ( R M S E ) + b}}$$

where m and b are real number coefficients determined from the FBV dataset and chosen to scale the NAS value to the 0-100 interval.

FIG. 20B illustrates a graph (1969a) of the time-varying point-by-point deviation between the measured and target FBV values for an exemplary NAS calculation. FIG. 20C illustrates a graph (1969b) of a sigmoid transformation function used to generate NAS values from the RMSE of the point-by-point deviation of FIG. 20B.

In some embodiments, normalized NAE values are impacted by noise in the raw NRM values. Data processing and/or filtering (e.g. low pass filtering, running window averages, etc.) may be beneficial to average over, subtract out or compensate for any such noise that is not relevant to therapy levels, e.g. pure measurement noise in activation estimates such as NRM or ECAP amplitudes. In various embodiments, data processing and/or filtering steps account for the effect of such processing on true effects such as (but not limited to) heartbeat-linked NRM variation.

In numerous embodiments, the one or more therapy quality metrics, such as NAE or NAS values, are used to provide a qualitative assessment of therapy outcome (e.g., as ‘poor’ quality or ‘high quality). High therapy error and/or variability tend to be associated with poorer quality outcomes. As such, error and viability metrics can be used as a predictor of higher quality patient therapy outcomes in open and closed loop therapies. Further, the ability to maintain stable closed loop therapy (i.e. keeping therapy error at low levels) can be used to predict high quality therapy outcomes.

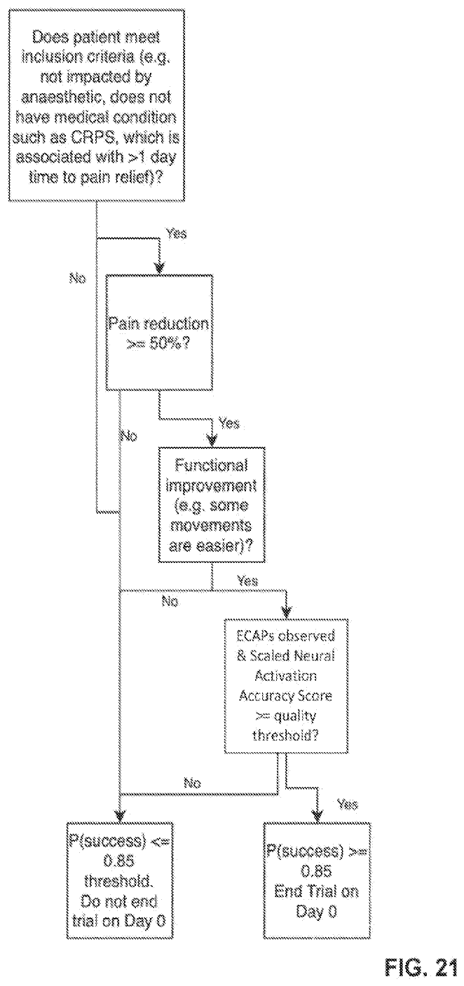

In a variety of embodiments, the SCS evaluation system considers additional information, such as a patient's medical background and/or demographic information, to calculate confidence scores. For example, patients who suffer from nociplastic pain/nociceptive pain or complex pain typically require more than 1 day to obtain pain relief. This may be used to downweight the significance of immediate therapeutic effect of the stimulation for the SCS assessment. FIG. 21 illustrates a decision-tree based process for an SCS trial scenario in accordance with an embodiment of the invention. It can readily be appreciated any number of different conditions and/or demographic classifications can have similar downweights (or upweights) as appropriate to the requirements of specific applications of the invention.

Confidence Scoring with Therapy Quality Metrics

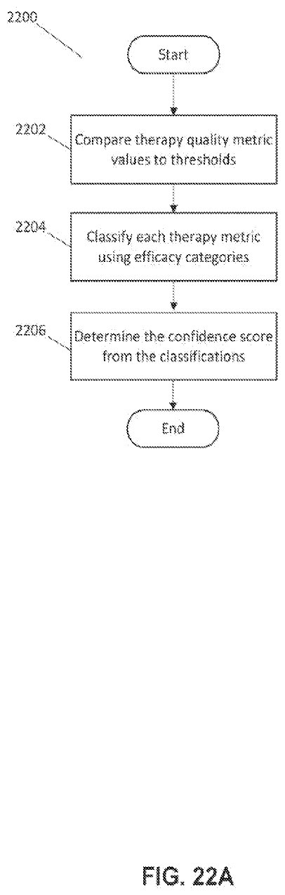

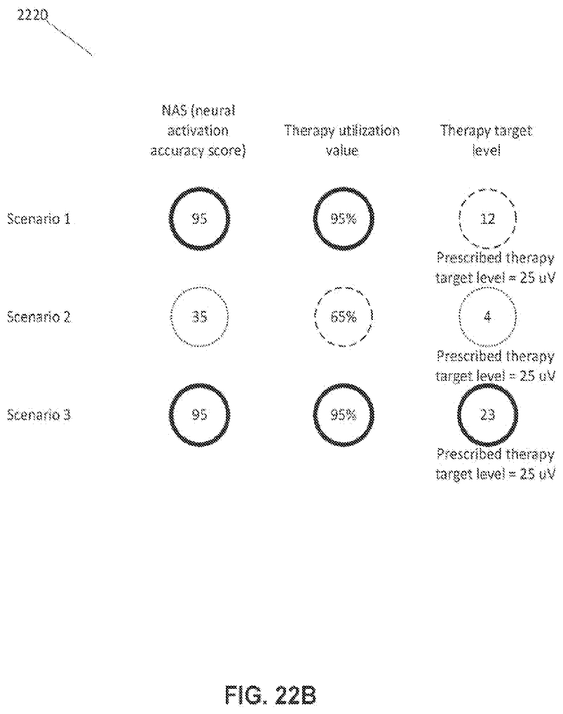

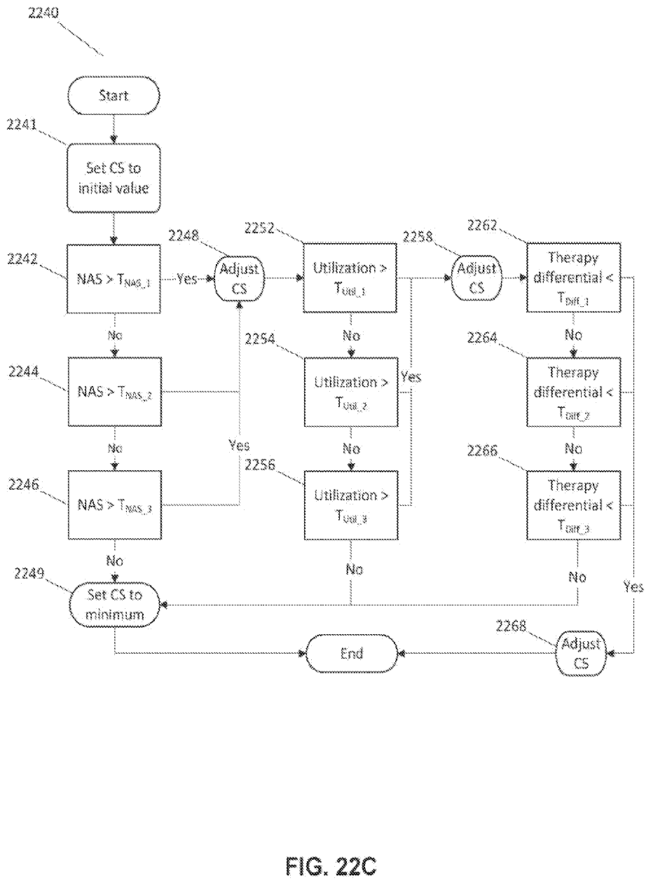

In the described embodiments, the confidence score is derived from the values of the evaluation metrics. In some examples, generation of the confidence score is specialized according to the one or more therapy quality metrics (if any) in the evaluation metric set. Values of the therapy quality metrics may be quantitatively translated or transformed into a confidence score by assigning, or classifying, the values of the therapy quality metrics in relation to a plurality of predetermined efficacy categories. FIG. 22A describes a process 2200 for determining the confidence score, including the steps of: comparing one or more therapy quality metric values to corresponding predetermined metric thresholds (2202): classifying each of the one or more therapy quality metric values as one of a plurality of predetermined efficacy categories (2204); and determining the confidence score based on the classifications of the one or more therapy quality metric values (2206).

In one example, the evaluation metrics comprise three therapy quality metrics including a therapy utilization value, a therapy target level value, and a neural activation accuracy score (NAS). FIG. 22B illustrates an exemplary classification table 2220, as rendered on a user interface of the SCS evaluator, showing the classification of these three therapy quality metric values. A three-tier classification system is used to determine the confidence score, where the value of each metric is indicated as belonging to one of the efficacy categories of ‘good’, ‘average’, or ‘bad’ (i.e., according to corresponding predetermined thresholds). The classifications according to the efficacy categories may be displayed in different outlines, as in the classification table 2220, e.g. a bold outline for ‘good’, a dashed outline for ‘average’, and a dotted outline for ‘bad’. NAS and therapy utilization values are scaled to the 0-100 interval, while the therapy target level is a raw potential value in microvolts (μV).

In scenario 1, the NAS and therapy utilization metrics are classified as ‘good’ however the therapy target level is classified as ‘average’ (i.e., since the prescribed activation level is 25 μV while the actual therapy target level is 12 μV). In scenario 2, the NAS and therapy target level metrics are both classified as ‘bad’ since the computed values have a ‘bad’ score and exhibit a large deviation from the prescribed target level, respectively. In scenario 3, the NAS and therapy utilization metrics are classified as ‘good’ and the therapy target level is also classified as ‘good’ due to the small difference between the prescribed therapy target level and the actual therapy target level (i.e., 2 uV).

Confidence scores may be calculated for each of the exemplary scenarios 1 to 3 by processing the NAS, utilization, and target level metrics. In some embodiments, the metric values, or corresponding confidence sub-scores, are processed according to a numerical formula to determine the confidence score. For example, the therapy target level value may be converted to a percentage sub-score based on the corresponding deviation from the prescribed target level, e.g.,

$$100 - 100 \times min ( 1 , \frac{\vert target - prescribed \vert}{prescribed} ) .$$

The (overall) confidence score may then be calculated as a weighted sum of the confidence sub-scores since all sub-scores are percentages in the 0-100 interval.

In other embodiments, the overall confidence score may be determined as one of a predetermined set of values. The predetermined set of confidence score values may include discrete real number values taken from the 0-100 interval, such as for example 100, 75, 50, 25, and 0. In such embodiments, the overall confidence score may be determined by selecting the closest predetermined confidence score value to a representative value (e.g., the weighted sum) of the therapy quality metric sub-scores.

In other embodiments, the confidence score is selected from the predetermined set of values based on a number of classifications of the therapy quality metric values in each efficacy category. In such embodiments, many different techniques may be implemented to select a predefined confidence score value from the efficacy classifications. For example, a predefined confidence score value of 100 may be selected if all three metrics are classified as ‘good’, a predefined confidence score value of 75 may be selected if two metrics are ‘good’ and the other ‘average’, a predefined confidence score value of 50 may be selected if all the metrics are ‘average’, etc.

In another example, the values of the metrics are translated into a confidence score using a decision-tree process. FIG. 22C illustrates a decision tree process 2240 for determining a confidence score by performing one or more evaluation tests against the one or more therapy quality metric values. At various steps of the process 2240, an evaluation test is conducted by the SCS evaluator involving performing a series of one or more comparisons between a therapy quality metric value and one or more corresponding threshold values (e.g., NAS test: 2242, 2244, 2246; therapy utilization test: 2252, 2254, 2256; therapy differential test: 2262, 2264, 2266). At various other steps (2248, 2258, 2268), the confidence score value is adjusted or set in response to the outcome of the one or more comparisons of each evaluation test.

In the described implementation, prior to commencing the testing of each therapy quality metric value, the evaluator sets the confidence score (CS) value to an initial predetermined value (2241). For example, the initial predetermined value may be ‘100’ representing the maximum possible confidence score obtainable from the process 2240. With reference to FIG. 22C, following initialization of the confidence score, the NAS metric value is tested against a set of thresholds (T_{NAS_1}, T_{NAS_2}, T_{NAS_3}) in steps 2242, 2244, and 2246. The threshold values are set such that T_{NAS_1}>T_{NAS_2}>T_{NAS_3}. Steps 2242, 2244 and 2246 are sequentially executed to perform a relative comparison between the NAS metric value and the thresholds.

In the described example, in response to the NAS metric value exceeding a respective one of the thresholds (T_{NAS_1}, T_{NAS_2}, T_{NAS_3}) the confidence score value is adjusted (2248). The adjustment of the confidence score is based on the value of the NAS metric relative to the thresholds of the test (i.e., T_{NAS_1}, T_{NAS_2}, T_{NAS_3}). For example, the adjustment may be in the form of a reduction in the current confidence score value, where the reduction is smaller if the NAS value exceeds T_{NAS_1} (at 2242) compared to if the NAS value exceeds T_{NAS_2} (at 2244) or T_{NAS_3} (at 2246). In some embodiments, the adjustment at step 2248 is optional depending on the test that was passed. For example, the current confidence score value may be maintained without adjustment if the NAS value exceeds T_{NAS_1}. Following the testing of the NAS metric, similar tests are performed for the therapy utilization metric value (2252, 2254, 2256) and the therapy differential value (2262, 2264, 2266) and consequent adjustments made to the current confidence score value (2258, 2268).

In the described implementation, in response to any of the NAS, therapy utilization metric or therapy differential values failing to exceed any of the thresholds in their respective test sequences, the evaluator is configured at step 2249 to set the confidence score value to a minimum value (e.g., ‘0)’) and terminate the process 2240. It will be appreciated that although three threshold values are used in the example described above, any arbitrary number of thresholds may be used to test the respective values of the therapy quality metrics.

The decision tree therapy quality metric evaluation process such as the process 2240 enables the dynamic variation of the confidence score (e.g., via adjustments to a predetermined initial value) based on whether the therapy quality metrics each exceed or fall below a set of corresponding expected or predetermined values (e.g., the test sequence thresholds T_{NAS_1}, T_{NAS_2}. T_{NAS_3} for the NAS metric). This is advantageous in that evaluation may be performed on each therapy quality metric value sequentially to prioritize a relative degree of importance of the therapy quality metrics as defined by the process tree, thereby enabling SCS efficacy or viability decisions to be made by terminating the evaluation early (e.g., if a test sequence of an important metric, which is evaluated in the tree before the sequence of one or more other less important metrics, indicates that the value of the important metric is unacceptably low). This also improves the computational efficiency of the SCS evaluator by preventing the execution of redundant data comparison steps in instances of early termination.

In some embodiments, the values of each therapy quality metric are processed to adjust one or more parameters of the SCS program to improve SCS effectiveness for the patient. The level or degree of adjustment may be determined by the values of each therapy quality metric and/or the overall confidence score. In one example, a deficiency in the value of a particular therapy quality metric compared to thresholds of the test sequence may be translated into an adjustment of a particular parameter of the SCS program (e.g., an increase to the prescribed target therapy level if the differential is too large). Adjustment may be performed iteratively by processing individual therapy quality metric values, determining the confidence score, adjusting the one or more parameters of the SCS program based on the confidence score (and/or the individual therapy quality metric values), and repeating the evaluation process until the confidence score reaches a desired value.

In some embodiments, the adjustment of the parameters of the SCS program is performed in conjunction with a decision-tree evaluation process such as process 2240. For example, the outputs of the test sequence comparisons (e.g., 2242, 2244, 2246; 2252, 2254, 2256; and 2262, 2264, 2266) may be used to adjust or otherwise set values of the SCS program parameters in addition, or as an alternative, to the confidence score value determined by the evaluation process.

Although specific systems and methods of spinal cord stimulator evaluation are discussed above, many different methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

LABEL LIST

| | |
|---|---|
| stimulator | 100 |
| patient | 108 |
| module | 110 |
| battery | 112 |
| telemetry module | 114 |
| controller | 116 |
| memory | 118 |
| clinical data | 120 |
| patient settings | 121 |
| control programs | 122 |
| pulse generator | 124 |
| electrode selection module | 126 |
| measurement circuitry | 128 |
| system ground | 130 |
| electrode array | 150 |
| current pulse | 160 |
| ECAPs | 170 |
| nerve | 180 |
| communications channel | 190 |
| external computing device | 192 |
| system | 300 |
| clinical settings controller | 302 |
| target ECAP controller | 304 |
| box | 308 |
| box | 309 |
| controller | 310 |
| box | 311 |
| stimulator | 312 |
| element | 313 |
| signal amplifier | 318 |
| ECAP detector | 320 |
| comparator | 324 |
| gain element | 336 |
| integrator | 338 |
| activation plot | 402 |
| ECAP threshold | 404 |
| comfort threshold | 408 |
| perception threshold | 410 |
| therapeutic range | 412 |
| activation plot | 502 |
| activation plot | 504 |
| activation plot | 506 |
| ECAP threshold | 508 |
| ECAP threshold | 510 |
| ECAP threshold | 512 |
| ECAP target | 520 |
| ECAP | 600 |
| neuromodulation system | 700 |
| neuromodulation device | 710 |
| remote controller | 720 |
| CST | 730 |
| clinical interface | 740 |
| charger | 750 |
| data flow | 800 |
| neuromodulation device | 804 |
| CPA | 810 |
| clinical data log file | 812 |
| CDV | 814 |
| clinical Data Uploader | 816 |
| database loader | 822 |
| database | 824 |
| data analysis web API | 826 |
| analysis module | 832 |
| system | 900 |
| SCS device | 910 |
| SCS evaluator | 920 |
| data server system | 930 |
| SCS evaluator | 1000 |
| processor | 1010 |
| input/output interface | 1020 |
| memory | 1030 |
| SCS evaluation application | 1032 |
| neural response data | 1034 |
| patient response metrics | 1036 |
| process | 1100 |
| process | 1100a |
| step | 1110 |
| step | 1120 |
| electrodes | 1130 |
| step | 1140 |
| step | 1150 |
| step | 1160 |
| step | 1170 |
| step | 1180 |
| step | 1190 |
| step | 1192 |
| step | 1194 |
| step | 1196 |
| process | 1400 |
| step | 1410 |
| step | 1420 |
| step | 1430 |
| step | 1440 |
| step | 1450 |
| step | 1460 |
| step | 1470 |
| process | 1500 |
| step | 1510 |
| step | 1520 |
| step | 1530 |
| step | 1540 |
| process | 1600 |
| step | 1610 |
| step | 1620 |
| step | 1630 |
| step | 1640 |
| step | 1650 |
| process | 1800 |
| step | 1810 |
| step | 1820 |
| step | 1830 |
| step | 1840 |
| step | 1850 |
| step | 1860 |
| step | 1870 |
| process | 1900 |
| step | 1910 |
| step | 1920 |
| step | 1930 |
| step | 1940 |
| step | 1950 |
| graph | 1951 |
| prescribed dose | 1952 |
| delivered dose | 1953 |
| process | 1960 |
| step | 1962 |
| step | 1964 |
| step | 1966 |
| step | 1968 |
| graph | 1969a |
| graph | 1969b |
| process | 2200 |
| step | 2202 |
| step | 2204 |
| step | 2206 |
| classification table | 2220 |
| process | 2240 |
| step | 2241 |
| step | 2242 |
| step | 2244 |
| step | 2246 |
| step | 2248 |
| step | 2249 |
| step | 2252 |
| step | 2254 |
| step | 2256 |
| step | 2258 |
| step | 2262 |
| step | 2264 |
| step | 2266 |
| step | 2268 |

### Examples of the Invention

Examples of various embodiments of the invention include:
1. A method of spinal cord stimulation evaluation, comprising: recording neural response data; calculating one or more metrics derived from a signal window of the neural response data; and using the one or more metrics to define a confidence score indicating the therapeutic effect likelihood of implantation of a spinal cord stimulator.
2. A spinal cord stimulation evaluator, comprising: a processor; and a memory, the memory containing a spinal cord stimulation evaluation application capable of directing the processor to: obtain neural response data generated by a spinal cord stimulator, where the neural response data describes a plurality of neural signals in the spinal cord of a patient; detect the presence of an evoked compound action potential (ECAP) in at least one neural signal in the plurality of neural signals; compute an ECAP likelihood score indicating the probability that the detection of the ECAP is accurate; compute a signal-to-noise ratio (SNR) of the ECAP:compute a signal-to-artefact ratio (SAR) of the ECAP:generate a confidence score based on the ECAP likelihood score, the SNR of the ECAP, and the SAR of the ECAP; and provide a determination that the spinal cord stimulator is likely to provide long-term therapeutic effect to the patient when the confidence score exceeds a threshold value.
3. A spinal cord stimulation evaluator, comprising: a processor; and a memory, the memory containing a spinal cord stimulation evaluation application capable of directing the processor to: obtain neural response data generated by a spinal cord stimulator, where the neural response data describes a plurality of neural signals in the spinal cord of a patient: detect the presence of an evoked compound action potential (ECAP) in at least one neural signal in the plurality of neural signals: compute a plurality of ECAP metrics based on the presence of the ECAP; and generate a confidence score based on the plurality of ECAP metrics: provide a determination that the spinal cord stimulator is likely to provide long-term therapeutic effect to the patient when the confidence score exceeds a threshold value.
4. The spinal cord stimulation evaluator of example 3, wherein the plurality of ECAP metrics comprises: a signal-to-noise ratio of the ECAP:a signal-to-artefact ratio of the ECAP; and a likelihood metric indicating the confidence with which the ECAP is detected.
5. The spinal cord stimulation evaluator of example 3, wherein the confidence score is further generated based on a second plurality of metrics, the second plurality of metrics comprising metrics selected from the group consisting of: a dermatome activation metric, a patient feedback metric, a gait metric, heart rate variability, pupil dilation, and action potential conduction velocity.
6. The spinal cord stimulation evaluator of example 3, wherein the confidence score is generated using a machine learning mode.
7. The spinal cord stimulation evaluator of example 3, wherein the confidence score is generated using a sum of the plurality of ECAP metrics.
8. A method of evaluating spinal cord stimulation as a viable therapy for a patient, comprising: obtaining neural response data generated by a spinal cord stimulator, where the neural response data describes a plurality of neural signals in the spinal cord of a patient; detecting the presence of an evoked compound action potential (ECAP) in at least one neural signal in the plurality of neural signals; computing a plurality of ECAP metrics based on the presence of the ECAP; and generating a confidence score based on the plurality of ECAP metrics; providing a determination that the spinal cord stimulator is likely to provide long-term therapeutic effect to the patient when the confidence score exceeds a threshold value.
9. The method of example 8, wherein the plurality of ECAP metrics comprises: a signal-to-noise ratio of the ECAP; a signal-to-artefact ratio of the ECAP; and a likelihood metric indicating the confidence with which the ECAP is detected.
10. The method of example 8, wherein the confidence score is further generated based on a second plurality of metrics, the second plurality of metrics comprising metrics selected from the group consisting of: a dermatome activation metric, a patient feedback metric, a gait metric, heart rate variability, pupil dilation, and action potential conduction velocity.
11. The method of example 8, wherein the confidence score is generated using a machine learning mode.
12. The method of example 8, wherein the confidence score is generated using a sum of the plurality of ECAP metrics.

1. A method of evaluating spinal cord stimulation (SCS), comprising:
obtaining neural response data generated by a spinal cord stimulator, where the neural response data describes one or more neural signals evoked in the spinal cord of a patient by applying diagnostic stimuli;
detecting one or more evoked compound action potentials (ECAPs) associated with the one or more neural signals respectively;
processing the one or more detected ECAPs to generate one or more evaluation metrics; and
using the one or more evaluation metrics to generate a confidence score indicating a degree of effectiveness of SCS on the patient.

2. The method of claim 1, wherein the confidence score indicates a likelihood that the SCS is effective to provide a therapeutic benefit to the patient.

3. The method of claim 1, further comprising, in response to the confidence score failing to exceed a predetermined threshold value, providing an indication of:
one or more additional activities to further assess whether the SCS is likely to provide a long-term therapeutic effect to the patient; or
an instruction or recommendation for explantation of the spinal cord stimulator, and/or one or more leads, from the patient.

4. The method of claim 1, further comprising, in response to the confidence score failing to exceed a predetermined threshold value:
determining one or more SCS parameters of an SCS program for adjustment; and
adjusting the determined one or more SCS parameters by an amount determined, at least in part, by processing the one or more evaluation metrics.

5. The method of claim 1, wherein the one or more evaluation metrics comprises one or more functionality metrics, the one or more functionality metrics including at least one of:
a signal-to-noise ratio of a given ECAP;
a signal-to-artefact ratio of the given ECAP; and
a likelihood metric indicating the confidence with which the given ECAP is detected, wherein the given ECAP is one of the detected ECAPs.

6. The method of claim 1, wherein the one or more evaluation metrics comprises a postural robustness metric.

7. The method of claim 6, wherein determining the postural robustness metric comprises:
(i) providing SCS to the patient via the spinal cord stimulator, wherein the patient is positioned in a candidate posture;
(ii) recording, in response to the SCS, a set of neural recruitment magnitudes (NRMs) of the detected ECAPs associated with the neural signals in the spinal cord of the patient in the candidate posture;
(iii) determining a first measure of variation to measure variation in values of the set of NRMs; and
(iv) computing a second measure of variation of a plurality of first measures of variation, wherein the plurality of first measures of variation are obtained by iteratively performing steps (i)-(iii) for different candidate postures.

8. The method of claim 1, wherein the one or more evaluation metrics comprises one or more therapy quality metrics, the one or more therapy quality metrics including at least one of:
a therapy utilization value;
a therapy target level value; and
a neural activation accuracy score (NAS).

9. The method of claim 8, wherein determining the NAS comprises:
(i) providing SCS to the patient via the spinal cord stimulator, wherein the patient is positioned in a candidate posture;
(ii) recording, in response to the SCS, a set of neural recruitment magnitudes (NRMs) of the detected ECAPs associated with the neural signals in the spinal cord of the patient in the candidate posture; and
(iii) processing the recorded set of NRMs to compute values of the NAS.

10. The method of claim 9, wherein the steps (i)-(ii) are iteratively repeated for different candidate postures such that step (iii) includes processing respective sets of NRMs recorded for the different candidate postures.

11. The method of claim 9, wherein computing values of the NAS comprises:
calculating a representative error value of each recorded set of NRMs relative to corresponding NRMs of a baseline target therapy level;
determining a neural activation error (NAE) value by processing one or more of the calculated representative error values; and
transforming the NAE value to generate a value of the NAS between zero and a predetermined maximum value.

12. The method of claim 11, wherein the representative error value of the set of NRMs is a root mean square error (RMSE) value.

13. The method of claim 12, wherein the NAE value is normalized or scaled relative to a predetermined feedback variable.

14. The method of claim 11, wherein the predetermined maximum value is 100.

15. The method of claim 8, wherein a confidence sub-score is generated for each of the one or more therapy quality metrics of the evaluation metrics.

16. The method of claim 15, wherein generating the confidence score comprises calculating a weighted sum of the confidence sub-scores.

17. The method of claim 8, wherein generating the confidence score comprises:
comparing one or more therapy quality metric values to one or more corresponding predetermined metric thresholds;
classifying each of the one or more therapy quality metric values as one of a plurality of predetermined efficacy categories based on the comparing; and
determining the confidence score based on the classifications of the one or more therapy quality metric values.

18. The method of claim 17, wherein the confidence score is determined as one of a set of predetermined confidence score values based on a number of classifications of the one or more therapy quality metric values in each efficacy category.

19. The method of claim 8, wherein generating the confidence score comprises:
performing one or more evaluation tests against the one or more therapy quality metric values, each evaluation test involving performing a series of one or more comparisons between a therapy quality metric value and one or more corresponding threshold values; and
setting or adjusting the confidence score in response to the outcome of the one or more comparisons of each evaluation test.

20. The method of claim 19, wherein the steps of performing evaluation tests and setting or adjusting the confidence score are organized according to a decision tree to prioritize a relative degree of importance of the therapy quality metrics.

21. The method of claim 19, wherein generating the confidence score further comprises, in response to a therapy quality metric value failing to exceed the corresponding threshold value, setting the confidence score to a minimum value.

22. The method of claim 1, wherein the confidence score is generated by further using one or more additional metrics selected from the group consisting of: a dermatome activation metric, a patient feedback metric, a gait metric, heart rate variability, and pupil dilation.

23. The method of claim 1, wherein the confidence score is generated using a machine learning mode.

24. The method of claim 1, wherein the confidence score is generated by calculating a sum of values of the one or more evaluation metrics.

25. A spinal cord stimulation evaluator, comprising:
a processor;
an input/output (I/O) interface configured to at least receive neural response data; and
a memory, the memory containing a spinal cord stimulation evaluation application configured to direct the processor to perform a method comprising:
obtaining the neural response data, where the neural response data indicates values of one or more neural signals evoked in the spinal cord of a patient by applying diagnostic stimuli;
detecting one or more evoked compound action potentials (ECAPs) associated with the one or more neural signals respectively;
processing the one or more detected ECAPs to generate one or more evaluation metrics; and
using the one or more evaluation metrics to generate a confidence score indicating a degree of effectiveness of SCS on the patient.

26. A system for evaluating spinal cord stimulation (SCS), comprising:
a stimulator device comprising an electrode array and a pulse generator, the stimulator device configured to:
apply, via the pulse generator, diagnostic stimuli to the spinal cord of a patient via one or more stimulus electrodes of the electrode array; and
measure, via one or more measurement electrodes of the electrode array, values of one or more neural response signals evoked by the diagnostic stimuli; and
at least one processor configured to:
receive neural response data indicating values of the one or more neural response signals;
detect one or more evoked compound action potentials (ECAPs) associated with the one or more neural signals respectively;
process the one or more detected ECAPs to generate one or more evaluation metrics; and
use the one or more evaluation metrics to generate a confidence score indicating a degree of effectiveness of SCS on the patient.

27. The system of claim 26, wherein the confidence score indicates a likelihood that the SCS is effective to provide a therapeutic benefit to the patient.

28. The system of claim 26, wherein the at least one processor is further configured to, in response to the confidence score failing to exceed a predetermined threshold value, provide an indication of:
one or more additional activities to further assess whether the SCS is likely to provide a long-term therapeutic effect to the patient; or
an instruction or recommendation for explantation of the spinal cord stimulator, and/or one or more leads, from the patient.

29. The system of claim 26, wherein the at least one processor is further configured to, in response to the confidence score failing to exceed a predetermined threshold value:
determine one or more SCS parameters of an SCS program for adjustment; and
adjust the determined one or more SCS parameters by an amount determined, at least in part, by processing the one or more evaluation metrics.

30. The system of claim 26, wherein the one or more evaluation metrics comprises one or more functionality metrics, the one or more functionality metrics including at least one of:
a signal-to-noise ratio of a given ECAP;
a signal-to-artefact ratio of the given ECAP; and
a likelihood metric indicating the confidence with which the given ECAP is detected,
wherein the given ECAP is one of the detected ECAPs.

31. The system of claim 26, wherein the one or more evaluation metrics comprises a postural robustness metric.

32. The system of claim 31, wherein determining the postural robustness metric comprises:
(i) providing, by the stimulator device, SCS to the patient, wherein the patient is positioned in a candidate posture;
(ii) recording, by the at least one processor and in response to the SCS, a set of neural recruitment magnitudes (NRMs) of the detected ECAPs associated with the neural signals in the spinal cord of the patient in the candidate posture;
(iii) determining, by the at least one processor, a first measure of variation to measure variation in the values of the set of NRMs; and
(iv) computing, by the at least one processor, a second measure of variation of a plurality of first measures of variation, wherein the plurality of first measures of variation are obtained by iteratively performing steps (i)-(iii) for different candidate postures.

33. The system of claim 26, wherein the one or more evaluation metrics comprises one or more therapy quality metrics, the one or more therapy quality metrics including at least one of:
a therapy utilization value;
a therapy target level value; and
a neural activation accuracy score (NAS).

34. The system of claim 33, wherein determining the NAS comprises:
(i) providing, by the stimulator device, SCS to the patient, wherein the patient is positioned in a candidate posture;
(ii) recording, by the at least one processor and in response to the SCS, a set of neural recruitment magnitudes (NRMs) of the detected ECAPs associated with the neural signals in the spinal cord of the patient in the candidate posture; and
(iii) processing, by the at least one processor, the recorded set of NRMs to compute values of the NAS.

35. The system of claim 34, wherein the steps (i)-(ii) are iteratively repeated for different candidate postures such that step (iii) includes processing respective sets of NRMs recorded for the different candidate postures.

36. The system of claim 34, wherein computing values of the NAS comprises:
calculating, by the at least one processor, a representative error value of each recorded set of NRMs relative to corresponding NRMs of a baseline target therapy level;
determining, by the at least one processor, a neural activation error (NAE) value by processing one or more of the calculated representative error values; and
transforming, by the at least one processor, the NAE value to generate a value of the NAS between zero and a predetermined maximum value.

37. The system of claim 36, wherein the representative error value of the set of NRMs is a root mean square error (RMSE) value.

38. The system of claim 37, wherein the NAE value is normalized or scaled relative to a predetermined feedback variable.

39. The system of claim 36, wherein the predetermined maximum value is 100.

40. The system of claim 33, wherein a confidence sub-score is generated for each of the one or more therapy quality metrics of the evaluation metrics.

41. The system of claim 40, wherein generating the confidence score comprises calculating a weighted sum of the confidence sub-scores.

42. The system of claim 33, wherein generating the confidence score comprises:
comparing, by the at least one processor, one or more therapy quality metric values to one or more corresponding predetermined metric thresholds;
classifying, by the at least one processor, each of the one or more therapy quality metric values as one of a plurality of predetermined efficacy categories based on the comparing; and
determining, by the at least one processor, the confidence score based on the classifications of the one or more therapy quality metric values.

43. The system of claim 42, wherein the confidence score is determined as one of a set of predetermined confidence score values based on a number of classifications of the one or more therapy quality metric values in each efficacy category.

44. The system of claim 33, wherein generating the confidence score comprises:
performing, by the at least one processor, one or more evaluation tests against the one or more therapy quality metric values, each evaluation test involving performing a series of one or more comparisons between a therapy quality metric value and one or more corresponding threshold values; and
setting or adjusting, by the at least one processor, the confidence score in response to the outcome of the one or more comparisons of each evaluation test.

45. The system of claim 44, wherein the steps of performing evaluation tests and setting or adjusting the confidence score are organized according to a decision tree to prioritize a relative degree of importance of the therapy quality metrics.

46. The system of claim 44, wherein generating the confidence score further comprises, in response to a therapy quality metric value failing to exceed the corresponding threshold value, setting, by the at least one processor, the confidence score to a minimum value.

47. The system of claim 26, wherein the confidence score is generated by further using one or more additional metrics selected from the group consisting of: a dermatome activation metric, a patient feedback metric, a gait metric, heart rate variability, and pupil dilation.

48. The system of claim 26, wherein the confidence score is generated using a machine learning mode.

49. The system of claim 26, wherein the confidence score is generated by calculating, by the at least one processor, a sum of the values of the one or more evaluation metrics.
